(19) United States
(12) Patent Application Publication
(10) Pub. No.: US 2024/0366229 A1
(43) Pub. Date: Nov. 7, 2024

(54) MAGNETIC DEVICES AND METHODS FOR MAGNETICALLY CLAMPING A PORTION OF A HOLLOW ORGAN OF THE DIGESTIVE TRACT

(71) Applicant: GT METABOLIC SOLUTIONS, INC., San Jose, CA (US)

(72) Inventor: Michel GAGNER, Montréal (CA)

(73) Assignee: GT METABOLIC SOLUTIONS, INC., San Jose, CA (US)

(21) Appl. No.: 18/650,852

(22) Filed: Apr. 30, 2024

Related U.S. Application Data
(60) Provisional application No. 63/499,310, filed on May 1, 2023.

(51) Int. Cl. A61B 34/00; A61B 17/00; A61B 17/122

(52) U.S. Cl. CPC A61B 17/122; A61B 2017/00823; A61B 2017/00876; A61B 2034/731

(57) ABSTRACT

There is provided a clamping device and method to resection an inwardly protruding portion present in a hollow organ of a patient, for example during bariatric surgeries, an appendectomy, a resection or excision surgery, etc. The clamping device includes a magnet assembly implantable into a lumen of the hollow organ of the patient. The magnet assembly has magnet elements arranged in two zones and is configured to be positioned around a base of the inwardly protruding portion. Opposing magnet elements are magnetically coupled together to compress opposite walls of the base therebetween until the walls are fused together, thereby partitioning the inwardly protruding portion from the remainder of the organ. The inwardly protruding portion can be pulled into the lumen of the hollow organ prior to the clamping device being positioned around the base. The inwardly protruding portion can be removed from the hollow organ to resection the organ.

### CROSS-REFERENCE TO RELATED APPLICATION

The present application claims priority from U.S. provisional patent application No. 63/499,310, filed on May 1, 2023, and entitled “MAGNETIC DEVICES AND METHODS FOR MAGNETICALLY CLAMPING A PORTION OF A HOLLOW ORGAN OF THE DIGESTIVE TRACT”, the disclosure of which is hereby incorporated by reference in its entirety.

### TECHNICAL FIELD

The technical field generally relates to devices and medical techniques for modifying a configuration of an organ. In particular, the technical field relates to devices and medical techniques for magnetically clamping a portion of an organ for reducing a volume of the organ or for removing a portion of the organ.

### BACKGROUND

Bariatric surgery procedures can be used to treat obesity and are generally aimed at restricting the size of an organ such as the stomach, and/or bypassing a portion of the stomach and/or the intestine. Examples of bariatric surgery procedures can include jejuno-ileal bypass, jejuno-colic shunt, biliopancreatic diversion, gastric bypass, Roux-en-Y gastric bypass, gastroplasty, gastric banding, vertical banded gastroplasty, silastic ring gastroplasty, and sleeve gastrectomy.

However, these bariatric surgery procedures often require alteration of the digestive tract through incisions, sutures, punctures and/or stapling, which can cause trauma to the organ being altered and lead to bleeding. Such bariatric surgery procedures can also lead to an increased risk of infection or other complications.

Similar complications can arise when resectioning an organ or when removing a portion of tissue or part of an organ. For instance, resection surgery procedures can be used to remove tissue or a part of an organ of the digestive system, endocrine system, and/or urinary system that is diseased or damaged. Examples of resection surgery procedures that can be performed on such organs can include appendectomy and tumour or lesion resections.

However, these resection surgical procedures often require alteration of the organ through cutting and removing the diseased or damaged tissue, which can cause trauma to the organ being altered and lead to excessive bleeding. Such resection surgical procedures can also lead to an increased risk of infection or other complications.

Accordingly, there remain a number of challenges with respect to surgical procedures that modify the configuration of an organ.

### SUMMARY

According to an aspect, there is provided a method for resectioning an inwardly protruding portion present in a hollow organ of a patient, the method comprising the steps of: inserting a magnet assembly of a clamping device into a lumen of the hollow organ, the magnet assembly comprising a first zone and a second zone, each of the first zone and the second zone comprising at least one magnet element having a magnetic-engaging side; positioning the magnet assembly at a base of the inwardly protruding portion such that the magnetic-engaging side of the first zone and the magnet-engaging side of the second zone face each other; magnetically coupling the first zone and the second zone together to compress opposite walls of the inwardly protruding portion therebetween; and allowing the opposite walls of the inwardly protruding portion to fuse together via ischemic pressure necrosis while the first zone and the second zone remain magnetically coupled during a healing time period.

In some implementations, the method further comprises pulling in a wall of the hollow organ into the lumen of the hollow organ to form the inwardly protruding portion.

In some implementations, inserting the magnet assembly of the clamping device into the lumen of the hollow organ comprises inserting the first zone of the magnet assembly first followed by the second zone of the magnet assembly.

In some implementations, positioning the magnet assembly at the base of the inwardly protruding portion comprises positioning the first zone along a first side of the base and positioning the second zone of the magnet assembly, adjacent to the first zone of the magnet assembly, along a second side of the base, opposed the first side.

In some implementations, positioning the magnet assembly at the base of the inwardly protruding portion further comprises coupling a first end and a second end of the magnet assembly with a releasable fastener to form an enclosed shape around the base.

In some implementations, the first zone and the second zone of the magnet assembly are separated by a transition zone, and the releasable fastener coupling the first end and the second end is provided in the transition zone.

In some implementations, the at least one magnet element of at least one of the first zone and the second zone comprises a plurality of magnet elements.

In some implementations, the at least one magnet element of both the first zone and the second zone each comprises a plurality of magnet elements.

In some implementations, the plurality of magnet elements are connected in series.

In some implementations, the plurality of magnet elements are flexibly connected in series.

In some implementations, the magnet-engaging side of the at least one magnet element of the first zone has a first zone magnetic pole, and the magnet-engaging side of the at least one magnet element of the second zone has a second zone magnetic pole that is different from the first zone magnetic pole.

In some implementations, magnetically coupling the first zone and the second zone together comprises magnetically coupling the first zone magnetic pole of the magnet-engaging side of the first zone with the second zone magnetic pole of the magnet-engaging side of the second zone.

In some implementations, the healing time period ranges from about 1 week to about 7 weeks.

In some implementations, the method further comprises removing the clamping device from the lumen of the hollow organ once the healing time period is completed.

In some implementations, the method further comprising removing the inwardly protruding portion from the lumen of the hollow organ once the healing time period is completed.

In some implementations, the hollow organ is a stomach, an esophagus, a small intestine, a large intestine, a gallbladder, a fallopian tube, or a urinary bladder of the patient.

In some implementations, the hollow organ is a stomach and the method for resectioning the inwardly protruding portion present in the stomach is a bariatric procedure.

In some implementations, the inwardly protruding portion comprises a tumor or a lesion.

In some implementations, the hollow organ is a large intestine of the patient and the inwardly protruding portion is an appendix of the patient.

In some implementations, inserting the magnet assembly of the clamping device into the lumen of the hollow organ is performed endoscopically.

According to another aspect, there is provided a method for resectioning an inwardly protruding portion present in a hollow organ of a patient, the method comprising the steps of: inserting a leading end of a magnet assembly of a clamping device into a lumen of the hollow organ; guiding the leading end to position the magnet assembly around a base of the inwardly protruding portion; magnetically coupling magnet elements of the magnet assembly together at the base of the inwardly protruding portion to compress opposite walls of the inwardly protruding portion therebetween.

In some implementations, the method further comprises pulling in a wall of the hollow organ into the lumen of the hollow organ to form the inwardly protruding portion.

In some implementations, the method further comprises allowing the opposite walls of the base to fuse together during a healing time period while the magnet elements are magnetically coupled together.

In some implementations, the method further comprises removing the clamping device from the lumen of the hollow organ once the healing time period is completed.

In some implementations, the method further comprises removing the inwardly protruding portion from the lumen of the hollow organ once healing time period is completed.

In some implementations, the clamping device further comprises a releasable fastener located at a trailing end of the magnet assembly, the releasable fastener being configured to releasably couple to the leading end of the magnet assembly.

In some implementations, first ones of the magnet elements are connected in series in a first zone and second ones of the magnet elements are connected in series in a second zone, the first zone and the second zone being opposed to each other once the magnet assembly is implanted in the hollow organ.

In some implementations, the first zone and the second zone are pivotally, elastically or flexibly connected to each other.

According to another aspect, there is provided a method for modifying the configuration of an organ of a patient, the method comprising the steps of: pulling in at least a portion of the organ into a lumen of an adjacent hollow organ of the patient to form an inwardly protruding portion; implanting a clamping device comprising a magnet assembly into the lumen of the adjacent hollow organ, the magnet assembly comprising: a first zone comprising a first magnet element; a second zone comprising a second magnet element; and a first transition zone extending between the first zone and the second zone; positioning the magnet assembly at a base of the inwardly protruding portion such that the first zone and the second zone face each other; magnetically coupling the first and second magnet elements together to compress opposite walls of the base; and allowing the opposite walls of the base to fuse together while the magnet elements remain magnetically coupled.

In some implementations, positioning the magnet assembly at the base of the inwardly protruding portion comprises releasably coupling the first and second zones in the transition zone to form an enclosed shape around the base.

According to another aspect, there is provided a clamping device for resectioning an inwardly protruding portion present in a lumen of a hollow organ of a patient, the clamping device comprising: a magnet assembly implantable into the lumen of the hollow organ of the patient, the magnet assembly comprises: a first zone comprising a first magnet element; and a second zone comprising a second magnet element; and the magnet assembly being configured to be positioned at a base of the inwardly protruding portion to magnetically couple the first zone and the second zone to compress opposite walls of the inwardly protruding portion therebetween during a healing time period.

In some implementations, the clamping device further comprises a transition zone extending between the first zone and the second zone, the transition zone comprising a fastener to couple a leading end of the first zone to a trailing end of the second zone.

In some implementations, the fastener comprises a flexible fastener.

In some implementations, the fastener comprises a releasable fastener.

In some implementations, the transition zone is configured to provide a pivotal transition between the first zone and the second zone.

In some implementations, the transition zone further comprises a flexible connector.

In some implementations, at least one of the first and second magnet elements comprises a plurality of magnet elements, the magnet elements of the plurality of magnet elements being connected to each other in series by a magnet element connector.

In some implementations, the magnet element connector is flexible.

In some implementations, the magnet element connector is a rigid or semi-rigid connector.

In some implementations, each of the first and second magnet elements comprises a magnet-engaging side magnetically couplable to each other.

In some implementations, the magnet-engaging side of the first magnet element has a first zone magnetic pole, and the magnet-engaging side of the second magnet element has a second zone magnetic pole that is different from the first zone magnetic pole.

In some implementations, the first and second magnet elements have a shape selected from the group consisting of an oblong shape, a stadium shape, a circular shape, a triangular shape, a rectangular shape, and an octagonal shape.

In some implementations, at least one of the first and second magnet elements comprise bevelled edges or rounded edges.

In some implementations, the clamping device is configurable between a pre-clamping configuration and a clamping configuration.

In some implementations, the pre-clamping configuration is an enclosed pre-clamping configuration or an uncoupled pre-clamping configuration.

In some implementations, at least one of the first and second magnet element comprises a housing comprising an organ-contacting side and being configured to receive the at least one of the first and second magnet element therein.

In some implementations, the organ-contacting side of the housing comprises an elongated flat contact surface.

In some implementations, the housing comprises bevelled edges.

In some implementations, the first and second magnet elements are received in a housing, the housing comprising a first end and a second end, and wherein the first end and the second end of the housing are releasably coupled to each other with a fastener.

In some implementations, the first and second magnet elements are received in a corresponding housing.

### BRIEF DESCRIPTION OF THE DRAWINGS

FIG. 1 is a side cross-sectional view schematic of a portion of a clamping device that includes a magnet assembly having a first zone and a second zone, the clamping device being shown in an extended pre-clamping configuration.

FIG. 2 is a side cross-sectional view schematic of a portion of a clamping device that includes a magnet assembly having a first zone and a second zone, the clamping device being shown in a looped pre-clamping configuration.

FIG. 3 is a perspective view schematic of a magnet element in an arbitrary spatial relationship with opposite walls of a base of an inwardly protruding portion of a hollow organ.

FIG. 4 is a side cross-sectional view schematic of a clamping device in an extended pre-clamping configuration, with an enlarged view of magnet elements in a first zone of a magnet assembly and magnet elements in a second zone of the magnet assembly.

FIG. 5 is a side cross-sectional view schematic of a clamping device in a looped pre-clamping configuration, with an enlarged view of magnet elements in a first zone of a magnet assembly and magnet elements in a second zone of the magnet assembly.

FIG. 6 is a front cross-sectional view schematic of a clamping device in an extended pre-clamping configuration shown for subsequent placement inside the lumen of the stomach.

FIG. 7 is a front cross-sectional view schematic of the clamping device shown in FIG. 6 and being folded inside the lumen of the stomach to be placed in a looped pre-clamping configuration.

FIG. 8 is a front cross-sectional view schematic of a surgical device drawing or pulling in a portion of the inner wall of the stomach into the lumen of the stomach.

FIG. 9 is a front cross-sectional view schematic of the clamping device shown in FIG. 7 in a loop pre-clamping configuration and being prepared to be positioned at a base of an inwardly protruding portion of the stomach.

FIG. 10 is a front cross-sectional view schematic of the clamping device shown in FIG. 9 being positioned at the base of the inwardly protruding portion in the lumen of the stomach in the looped pre-clamping configuration.

FIG. 11 is a perspective view schematic of a magnet element shown in an arbitrary spatial relationship relative to opposite walls of a base of an inwardly protruding portion, the magnet element being received in a corresponding housing.

FIG. 12 is a perspective view schematic of three magnet elements of a portion of a magnet assembly shown in an arbitrary spatial relationship relative to opposite walls of a base of an inwardly protruding portion, the magnet elements being received in a single housing.

FIG. 13 is a perspective view schematic of three magnet elements of a portion of a magnet assembly shown in an arbitrary spatial relationship relative to opposite walls of a base of an inwardly protruding portion, the magnet elements being received in a corresponding housing having rounded edges, the successive housings being flexibly joined together by a thinner extension of the housing.

FIG. 14 is a perspective view schematic of three magnet elements of a portion of a magnet assembly shown in an arbitrary spatial relationship, adjacent magnet elements being flexibly joined together by a flexible connector.

FIG. 15 is a perspective view schematic of three magnet elements of a portion of a magnet assembly shown in an arbitrary spatial relationship, each magnet element being received in a corresponding housing, and adjacent magnet elements being flexibly joined together by a flexible connector extending between corresponding housings.

FIG. 16 is a perspective view schematic of three magnet elements of a portion of a magnet assembly shown in an arbitrary spatial relationship, the magnet elements being received in a corresponding housing, and the magnet elements being flexibly joined together within the housing by a flexible connector extending between adjacent magnet elements.

FIG. 17 is a perspective view schematic of three magnet elements of a portion of a magnet assembly shown in an arbitrary spatial relationship, each magnet element having rounded edges, and adjacent magnet elements being flexibly joined together by a flexible connector.

FIG. 18 is a perspective view schematic of six magnet elements of a portion of a magnet assembly shown in an arbitrary spatial relationship, each magnet element being received in a corresponding housing, the housing having bevelled edges, adjacent magnet elements being flexibly joined together by a flexible connector extending between corresponding housings, and the magnet elements being shown facing each other.

FIG. 19 is a side cross-sectional view schematic of a clamping device positioned at the base of the inwardly protruding portion of the stomach shown in FIG. 10 taken at line 19-19, the clamping device having six magnet elements of a portion of a magnet assembly shown in an arbitrary spatial relationship, each magnet element having bevelled edges, adjacent magnet elements being flexibly joined together by a flexible connector, the magnets being shown with walls of the base of the inwardly protruding portion therebetween.

FIG. 20 is a top perspective view schematic of an enclosed clamping device that includes two magnet elements in a first zone and two magnet elements in a second zone and the first and second zones are connected to each other at both ends thereof with a flexible connector.

FIG. 21 is a top perspective view schematic of an enclosed clamping device that includes a plurality of magnet elements in a first zone and a plurality of magnet elements in a second zone, the first and second zones being connected to each other at a first transition zone with a flexible connector and at a second transition zone with a releasable fastener.

FIG. 22 is a top perspective view schematic of an enclosed clamping device that includes three magnet elements in a first zone and three magnet elements in a second zone and the first and second zones are connected to each other at both ends thereof with a releasable fastener.

FIG. 23 is a side cross-sectional view schematic of a clamping device that includes magnet elements received in a housing having an elongated flat contact surface on an organ-contacting side thereof, the clamping device being shown in a clamping configuration with opposite walls of a base of an inwardly protruding portion being compressed between the elongated flat contact surfaces.

FIG. 24 is a top view schematic of a clamping device that includes magnet elements, the magnet elements being arranged to provide a curved region along the length of the portioning device.

FIG. 25A is a front cross-sectional view schematic of a stomach, showing a surgical tool grasping and pulling in a portion of the wall of the stomach within the lumen of the stomach.

FIG. 25B is a front cross-sectional view schematic of the stomach shown in FIG. 25A, showing the inwardly protruding portion before a clamping device is placed at a clamp line located at a base of an inwardly protruding portion in the lumen of the stomach from a remaining portion of the stomach.

FIG. 25C is a front cross-sectional view schematic of the stomach shown in FIG. 25A, showing the inwardly protruding portion after or during the clamping device being positioned along the clamp line located at the base of the inwardly protruding portion in the lumen of the stomach from the remaining portion of the stomach.

FIG. 26A is a front cross-sectional view schematic of a stomach, showing an inwardly protruding portion being drawn or pulled into the lumen from the inner wall of the stomach.

FIG. 26B is a front cross-sectional view schematic of the stomach shown in FIG. 26A, showing an inwardly protruding portion before a clamping device is placed at a clamp line located at a base of an inwardly protruding portion in the lumen of the stomach from a remaining portion of the stomach.

FIG. 26C is a front cross-sectional view schematic of the stomach shown in FIG. 26A, showing the inwardly protruding portion after or during the clamping device being positioned along the clamp line located at the base of the inwardly protruding portion in the lumen of the stomach from the remaining portion of the stomach.

FIG. 27A is a front cross-sectional view schematic of a portion of an abdominal cavity with a large intestine, small intestine, and appendix, showing a surgical tool grasping and pulling in the appendix inwardly into the large intestine.

FIG. 27B is a front cross-sectional view schematic of the abdominal cavity shown in FIG. 27A, showing the clamp line at a base of the appendix as the inwardly protruding portion in the lumen of the large intestine after a clamping device is placed along the clamp line.

FIG. 28 is a front cross-sectional view schematic view of a bladder of a female with a tumour on an interior wall of the lumen of the bladder, showing a clamp line.

FIG. 29A is a front cross-sectional view schematic of a bladder of a female with a tumour on an exterior wall of the bladder, showing a surgical tool configured for grasping and pulling in a portion of the wall of the lumen of the bladder.

FIG. 29B is a front cross-sectional view schematic of the bladder shown in FIG. 29A, showing an inwardly protruding portion before a clamping device is placed along a clamp line located at a base of the inwardly protruding portion.

FIG. 29C is a front cross-sectional view schematic of the bladder shown in FIG. 29A, showing the inwardly protruding portion after or during the clamping device being positioned along the clamp line located at the base the inwardly protruding portion.

### DETAILED DESCRIPTION

Surgical procedures to treat various medical conditions can include modifying the configuration of an organ, such as the esophagus, stomach, gallbladder, a duct of the biliary system, small intestine, colon, gallbladder, fallopian tube, the appendix, or urinary bladder. Modifying the configuration of an organ can include reducing the size of the lumen of a hollow organ, or removing a portion of a solid organ via an adjacent hollow organ. Such surgical procedures can be performed for instance in the context of bariatric surgeries or other digestive surgeries such as those that can be performed as part of a cancer treatment, resection surgeries, etc. Modifying the configuration of an organ by reducing the size of the lumen of the organ or by removing a portion of the organ can include grasping and pulling in a portion of the organ to obtain an inwardly protruding portion, and clamping opposite walls of the inwardly protruding portion at a base of the inwardly protruding portion using a magnetic clamping device to fuse together the opposite walls of the base of the inwardly protruding portion. In some implementations, the inwardly protruding portion can be removed in a secondary procedure, or the inwardly protruding portion can be naturally excreted from the patient. The clamping device may include various features. It is to be understood that as used herein, the expression “clamping device” can be used interchangeably with the expression “clamping implant” throughout the present description, as the clamping device is configured to be implanted and remain a certain period of time, which can extend from days to weeks, within the lumen of the target hollow organ of the patient.

The clamping device can include a flexible magnet assembly that can be inserted into the lumen of the target hollow organ or into the lumen of an adjacent hollow organ of the patient. The portion to be removed or resectioned can then be drawn or pulled in to form an inwardly protruding portion and the clamping device can be positioned at a base of the inwardly protruding portion in the lumen of the organ or in the lumen of the adjacent hollow organ. The flexibility of the magnet assembly can be conferred by flexibly connecting a series of magnet elements together to form a train of magnet elements. Each one of the magnet elements includes a respective magnet-engaging side, and consecutive ones of magnet elements have their respective magnet-engaging side on the same side. In some implementations, one or more magnet elements can be received in a respective housing, or the series of magnet elements can be received together in a single housing.

The clamping device can be configured to adopt a pre-clamping configuration and a clamping configuration. The pre-clamping configuration can facilitate the insertion of the clamping device into the lumen of the target hollow organ of a patient, and subsequent placement at the base of the inwardly protruding portion of the target hollow organ. The pre-clamping configuration can thus be a configuration that can enable the clamping device to be placed at the base of the inwardly protruding portion of the hollow organ, and can take the form of a loop, circle or band, while the clamping configuration can be a configuration that can enable the clamping of the inwardly protruding portion.

In the pre-clamping configuration, the magnet-engaging sides of the plurality of magnets are magnetically uncoupled to each other. To achieve such a pre-clamping configuration, the magnet assembly can extend longitudinally such that the magnet-engaging sides are not facing each other and can be inserted into the lumen of the target hollow organ longitudinally, e.g., as a string. This type of pre-clamping configuration can be referred to as an extended pre-clamping configuration. In another example of pre-clamping configuration, the magnet assembly can form a U-shape to obtain a partial or closed loop with opposite magnet-engaging sides of the magnet elements facing each other, but being far enough from each other that the opposite magnet-engaging sides of the magnet elements remain magnetically uncoupled so as to allow the partial or closed loop to be placed at the base of the inwardly protruding portion. This pre-clamping configuration can be referred to as a looped pre-clamping configuration. In other configurations, the magnet assembly can form an enclosed shape, such that opposing magnet-engaging sides of the magnet elements are facing each other, but are far enough from each other such that the opposite magnet-engaging sides remain magnetically uncoupled so as to allow the enclosed shape to be placed at the base of the inwardly protruding portion. This pre-clamping configuration can be referred to as an enclosed pre-clamping configuration. In other configurations, the magnet assembly can have two zones of magnet elements that are releasably coupled to each other, such that when uncoupled, the magnet assembly can comprise two portions that can be placed at the base of the inwardly protruding portion and then coupled at one or both ends. This pre-clamping configuration can be referred to as an uncoupled pre-clamping configuration.

The choice of pre-clamping configuration can depend on the target hollow organ or the inwardly protruding portion around which the magnet assembly will be placed, and can take into consideration the space available within the lumen of the target hollow organ. If the magnet assembly is initially inserted as a string, the pre-configuration will eventually include the formation of the U-shape or enclosed shape for placement around the inwardly protruding portion while the magnet elements remain magnetically uncoupled. In addition to facilitating the insertion of the clamping device into the lumen of the target hollow organ, the pre-clamping configuration can facilitate placement of the magnet assembly intra-luminally around the inwardly protruding portion of the hollow target hollow organ along a given clamp line. In the context of the present description, the clamp line corresponds to a delimitation between the inwardly protruding portion and the remaining portion of the target hollow organ, and thus to a tracing where the magnet-engaging sides of the magnet assembly are placed against to clamp the inwardly protruding portion from a remainder portion of the target hollow organ. The clamp line also corresponds to the base of the inwardly protruding portion (i.e., where the inwardly protruding portion is delineated from the remainder portion of the organ).

Once the clamping device is placed at the base of the inwardly protruding portion of the hollow organ, the magnet assembly can be placed in the clamping configuration. In the clamping configuration, opposite magnet elements on each side of the target hollow organ are placed in sufficiently close proximity so that opposite magnet-engaging sides of the magnet elements facing each other are magnetically coupled to bring together and compress portions of the wall of the target hollow organ therebetween (i.e., the portions of wall at the base of the inwardly protrusion portion), without having to puncture the tissue of the organ. The magnetic coupling maintains the magnet elements in place, and the pressure applied by the magnet elements on the portions of the wall of the target hollow organ triggers a healing mechanism that over time, can lead to the fusion of the portions of the wall of the organ along the edges of the magnet elements. In the context of the present description, the term “fusion” can be interpreted as corresponding to the resulting healed tissue on the edges of the magnet elements that are now joined to form a single structure. The portions of the wall of the organ that were initially compressed between the magnet-engaging sides along the portioning line are eventually subjected to ischemic pressure necrosis, and after a certain period of time, referred to herein as a healing time period, which can extend from days to weeks, a space corresponding substantially to the width of the magnet elements will end up being defined at the base of the inwardly protruding portion, thereby separating the inwardly protruding portion from the remaining portion of the hollow organ.

The clamping device is configured to remain in the lumen of the target hollow organ of the patient for a period of time that is sufficient to allow the healing process to take place and for the fusion of the portions of the wall of the organ on the edges the magnet elements to occur. Monitoring of the healing process can be advantageous to determine at which moment it may be advisable to remove the clamping device from the lumen of the target hollow organ of the patient after the fusion of the tissues has occurred.

The clamping device can also include additional features. For instance, the clamping device can include one or more features that can contribute to aid in the placement of the magnet assembly within the lumen of the target hollow organ. Such features can include a flexible elongated member, such as a flexible cord or a flexible wire, that is configured to extend from one extremity of the magnet assembly and that can also be referred to as leading elongated member. The flexible elongated member can be configured for engagement with a delivery catheter, and in turn, the delivery catheter can be used to carry and navigate the magnet assembly via the leading elongated member into the lumen, and at the base of the inwardly protruding portion of the target hollow organ.

Optionally, the clamping device can also include a second flexible elongated member at the other extremity of the magnet assembly, opposite the leading elongated member, and this second flexible elongated member can be referred to as trailing elongated member. The trailing elongated member can be useful during implantation of the magnet assembly to aid in the placement of the magnet assembly.

Various implementations of the clamping device and associated methods will now be described in greater detail.

### Magnet Assembly

### Magnet Elements

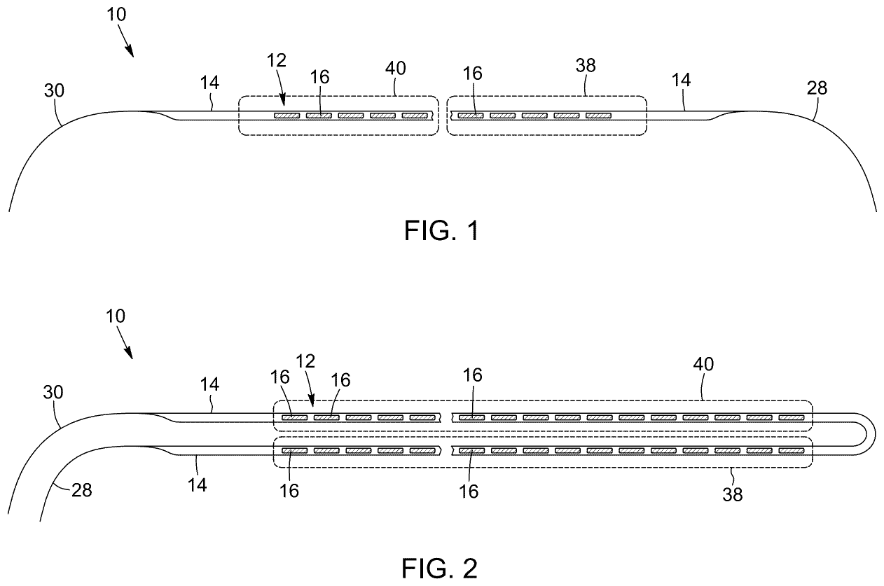

With reference to FIGS. 1 to 5, an implementation of a clamping device 10 is shown. The clamping device 10 includes a magnet assembly 12 that is received in a housing 14. The magnet assembly 12 includes a plurality of magnet elements 16 that are connected together, or joined, in a flexible manner. The magnet element 16 includes a top side 18, a bottom side 20, lateral sides 22 and longitudinal sides 24. In the context of the present description, the top side 18 of the magnet element 16 refers to the side that faces the organ tissue 26 at the base of the inwardly protruding portion once the magnet assembly 12 is implanted in the lumen of the target hollow organ of the patient and placed at the base of the inwardly protruding portion. In the implementation shown, the clamping device 10 further includes a flexible elongated member at each extremity, i.e., a leading elongated member 28 and a trailing elongated member 30. In addition, the housing 14 in which the magnet elements 16 are received is also flexible. It is to be understood that although the leading elongated member 28 and a trailing elongated member 30 are shown in FIGS. 1, 2, 4-7, 9, 10, 23 and 24, the leading elongated member 28 and a trailing elongated member 30 can be omitted of the clamping device 10. In other words, the leading elongated member 28 and a trailing elongated member 30 are optional features of the clamping device 10 described herein.

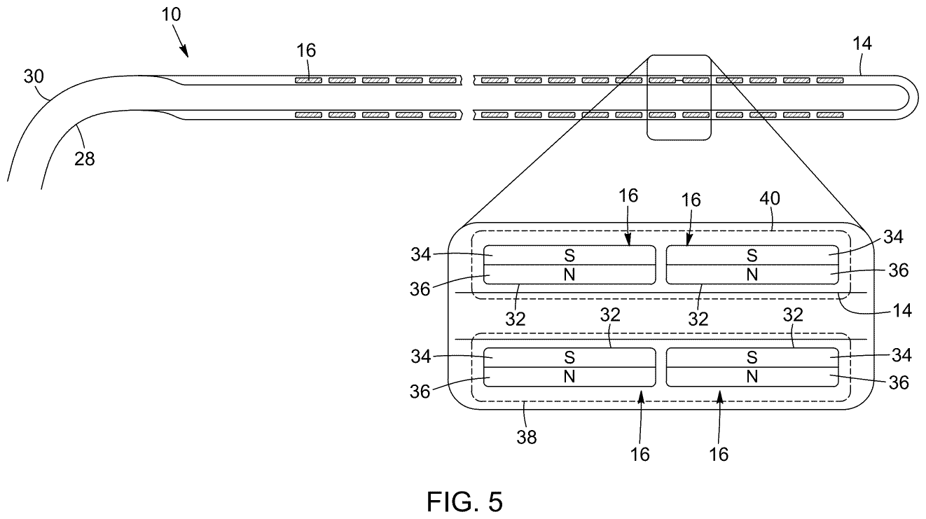

In FIG. 1, the clamping device 10 is in a pre-clamping configuration, where the clamping device 10 is extended longitudinally such that the extremities of the magnet assembly 12 are spaced apart and opposite to each other. This type of pre-clamping configuration can be referred to as an extended pre-clamping configuration. In FIGS. 2 and 5, the clamping device 10 is also in a pre-clamping configuration, albeit one that can be referred to as a looped pre-clamping configuration. In the looped pre-clamping configuration, the magnet assembly 12 is folded on itself in a U-shape to form a partial or closed loop, depending on the intended application. The choice of the pre-clamping configuration for the magnet assembly 12 can depend on the hollow organ that is targeted to treat, and the choice of insertion method that is considered. As mentioned above, if the magnet assembly 12 is initially inserted as a string such as in shown in FIG. 1, the pre-clamping configuration eventually includes the formation of the U-shape in a partial or closed loop for placement at the base of the inwardly protruding portion of the target hollow organ while the magnet elements 16 remain magnetically uncoupled. In other words, one of the characteristics of the pre-clamping configuration of the magnet assembly 12 is that the magnet elements 16 are magnetically uncoupled, and the magnet assembly 12 can adopt various configurations where the magnet elements 16 are magnetically uncoupled. In addition to facilitating the insertion of the clamping device 10 into the lumen of the target hollow organ, the pre-clamping configuration can also facilitate placement of the magnet assembly 12 at the base of the inwardly protruding portion in the lumen of the target hollow organ and along a given clamp line 42. Additional details regarding these considerations are provided below.

The magnet elements 16 of the magnet assembly 12 can be any type of suitable magnet. In some implementations, the magnet elements 16 can be chosen according to their attractive force, i.e., according to the pressure that will be exerted on the surface area of the tissue that will eventually be compressed between magnetically coupled magnet elements 16. Factors influencing the attractive force of the magnet element 16 can include the shape of the magnet element 16, the thickness of the magnet element 16, the material that the magnet element 16 is made of, etc. Examples of materials can include neodymium magnets (e.g., NdFeB magnets), rare earth magnets and ferrite magnets.

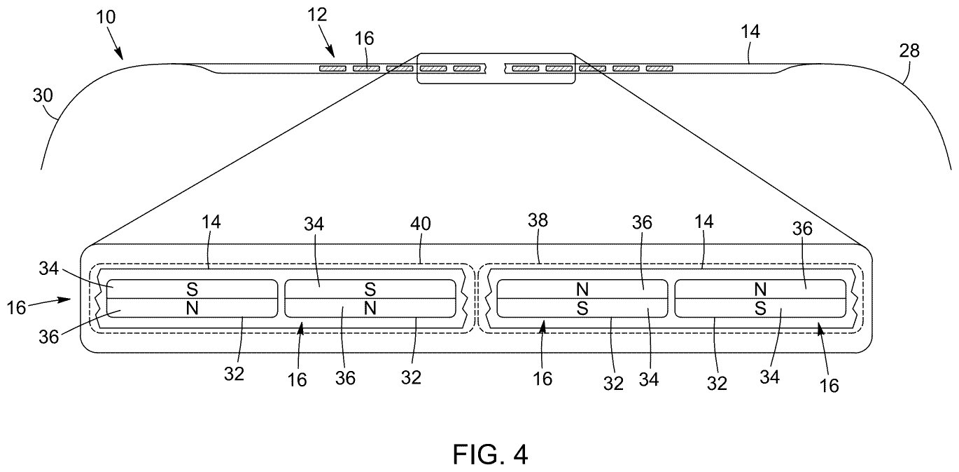

With specific reference to FIGS. 4 and 5, each one of the magnet elements 16 can be a dipole magnet having magnetic poles across the thickness of the magnet element 16, such that the magnet element 16 includes a first magnetic pole 34 on one side thereof and a second magnetic pole 36 on another side thereof, the second magnetic pole 36 being different than the first magnetic pole 34. One side of the magnet element 16 corresponds to a magnet-engaging side 32, which is the side of the magnet element 16 that will eventually face the internal surface of the organ tissue 26 of the hollow organ to be treated once the magnet assembly 12 is installed at the base of the inwardly protruding portion 50 of the target hollow organ, which is also referred to above as the top side 18 of the magnet element 16.

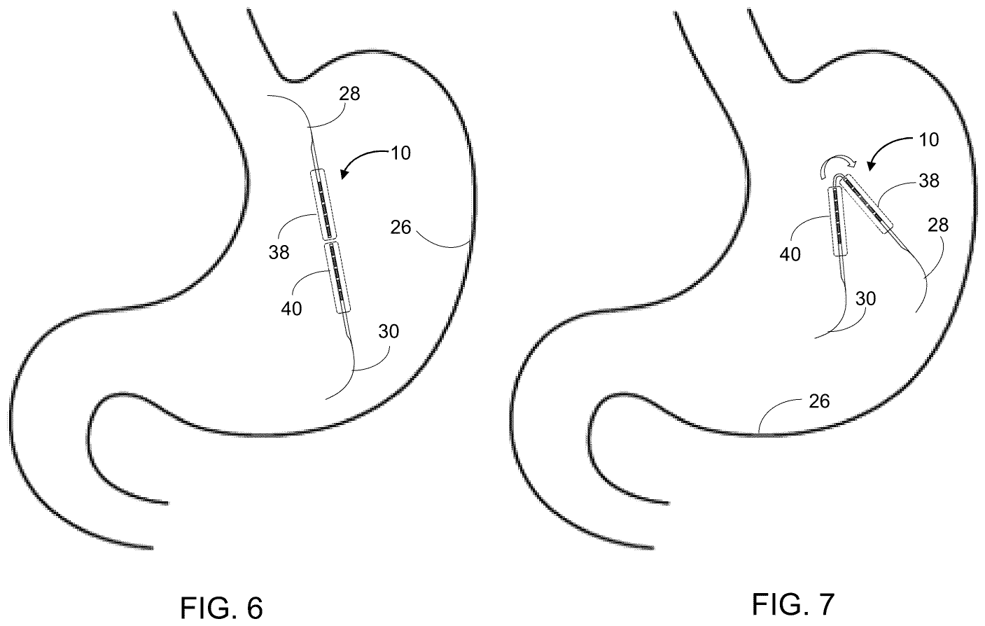

The magnet elements 16 can be positioned strategically along the length of the magnet assembly 12 to obtain a desired functionality of the magnet assembly 12. With reference to FIGS. 1 to 10, a first zone 38 and a second zone 40 of the magnet assembly 12 are represented. The first zone 38 of the magnet assembly 12 corresponds to a portion of the magnet assembly 12 that can be the first to be inserted into the lumen of the hollow organ, when the clamping device 10 is in an extended pre-clamping configuration such as in FIG. 1, by guiding a leading end or, in some instances, the leading elongated member 28, into the lumen, such as in FIG. 6. Then, the first zone 38 of the magnet assembly 12 would be the one that is flipped around a base or cuspidal point of the inwardly protruding portion 50 of the target hollow organ, for example in a posterior position, such as shown in FIGS. 7 and 10. The base of the inwardly protruding portion 50 is the area along the resection or clamping line 42 where the organ tissue 26 is brought together in close contact to form the inwardly protruding portion 50. In some implementations, the organ tissue 26 can be an inner or interior wall tissue of the lumen of the target hollow organ. For example, when the wall of the target hollow organ comprises more than one type of tissue, the organ tissue 26 being compressed by the clamping device 10 can include only the inner-most (lumen facing) tissue, or can include the first and second inner-most layers. In other implementations, the organ tissue 26 being compressed can include all layers of the target hollow organ wall, such as shown in FIG. 10. In some instances, for example during an appendectomy or a tumor resection procedure, the organ tissue 26 facing the magnet-engaging sides 32 of the magnet elements 16 of the magnet assembly 12 can include the appendix and tumor itself, respectively, the tumor and the appendix each corresponding to an inwardly protruding portion.

In the implementation shown in FIGS. 6 to 10, the second zone 40 corresponds to a portion of the magnet assembly 12 that follows the first zone 38 when the magnet assembly 12 is inserted into the lumen of the hollow organ. The second zone 40 can remain in an anterior position without being flipped around to the posterior side of the outer surface of the base of the inwardly protruding portion, such as shown in FIGS. 7 and 10. It is to be understood that in other implementations, for example when the base of the inwardly protruding portion is not located on a side portion of the hollow organ but rather on an anterior side or a posterior side thereof, the first zone 38 and the second zone 40 can be considered as being provided side-by-side once placed at the base of the inwardly protruding portion, such as anteriorly or posteriorly, laterally or medially, or inferiorly and superiorly.

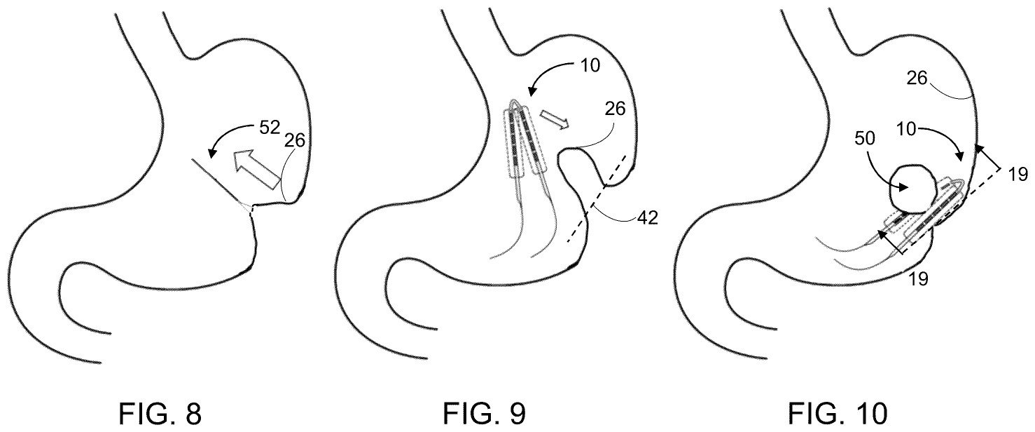

Referring to FIGS. 8 to 10, it is also contemplated that the magnet assembly 12 can be inserted into the lumen of the target hollow organ in a looped pre-clamping configuration, such that the first and second zones 38, 40 of the magnet assembly 12 can be navigated in the lumen side-by-side rather than being extended.

Referring back to FIGS. 4 and 5, the first zone 38 includes a series of magnet elements 16 that each has a magnet-engaging side 32 having a same magnetic pole among each other (i.e., a first zone magnetic pole), and the second zone 40 includes a series of magnet elements 16 that also each have a magnet-engaging side 32 having a same magnetic pole among each other (i.e., a second zone magnetic pole), but that is different from the magnetic pole of the magnet-engaging side 32 of the magnet elements 16 of the first zone 38. The magnet-engaging side 32 corresponds to the top side 18 of the magnet element 16 described above, i.e., the side that faces the interior walls of the hollow organ. For instance, as shown in FIG. 4, the clamping device 10 is in an extended pre-clamping configuration, and the second zone 40 of the magnet assembly 12 is longitudinally spaced apart from the first zone 38. The magnet-engaging side 32 of the magnet elements 16 of the first zone 38 has a North pole, and the magnet-engaging side 32 of the magnet elements 16 of the second zone 40 has a South pole. Similarly, in FIG. 5, the clamping device 10 is in a looped pre-clamping configuration, and the magnet-engaging side 32 of the magnet elements 16 of the first zone 38 has a North pole, and the magnet-engaging side 32 of the magnet elements 16 of the second zone 40 has a South pole.

This configuration of the magnet assembly 12, with the magnet elements 16 having a different magnet-engaging side 32 depending on the zone 38, 40 that they are in along the magnet assembly 12, enables the magnet elements 16 of the first zone 38 and the second zone 40 of the magnet assembly 12 to attract each other and magnetically couple once the magnet assembly 12 is in the clamping configuration.

As mentioned above, the magnet elements 16 can have various shapes and sizes. The choice of the shape and/or the size of the magnet elements 16 can depend on the target hollow organ within which the clamping device 10 will be implanted and the size of the portioned to be resectioned (i.e., the size of the inwardly protruding portion 50). For instance, when a large section of an organ like the stomach, larger or elongated magnet elements 16 can be used as part of the magnet assembly 12. On the other hand, the length of the magnet element 16 can remain sufficiently small to permit the navigation of the magnet assembly 12 around the outer surface of the base of the inwardly protruding portion 50, or to facilitate the insertion of the magnet assembly 12 into the lumen of the target hollow organ. For instance, in some implementations, magnet elements 16 designed to remove the appendix through the lumen of the large intestine may have a size ranging from about 5 mm to about 40 mm, with the total length of each of the first and second zones 38, 40 totaling between about 10 mm and about 50 mm, or between about 20 mm and 40 mm; whereas magnet elements 16 designed to resection an inwardly protruding portion 50 of a stomach may have a size ranging from about 5 mm to about 100 mm, with the total length of each of the first and second zones 38, 40 totaling between about 30 mm and about 100 mm, or about 80 mm. It is to be understood that these examples are given for illustrative purposes only, and should not be interpreted restrictively.

It is also be understood that the magnet assembly 12 can have any number and/or size of magnet elements 16. For example, a magnet assembly 12 designed to remove the appendix can include a first zone 38 with a single magnet element 16 that is about 30 mm and a second zone 40 with a single magnet element 16 that is also about 30 mm. Alternatively, the first and second zones 38, 40 can each comprise multiple magnet elements 16, such as six magnet elements 16 each having a length of about 5 mm, for instance, or the magnets can have a different length from one another. In yet other implementations, the first zone 38 can include a single magnet of a given length, and the second zone 40 can include multiple magnets of a same length or a different length. Similar considerations apply to a magnet assembly 12 designed to resection the stomach. For instance, a magnet assembly 12 designed to resection the stomach can include first and second zones 38, 40 that each have a single magnet element 16, which can, for example, have a length of about 80 mm, or can include any number of magnet elements 16.

As a general relationship between the size of the magnet elements 16 and the lumen of the target hollow organ, magnet elements 16 designed to resection an inwardly protruding portion 50 of a hollow organ that has a thicker wall and/or that is considered as having a large lumen can generally be larger than magnet elements 16 designed for a hollow organ that has a smaller lumen and/or a thinner wall. This general relationship takes into consideration the principle that larger and/or thicker hollow organs may benefit from a larger attractive force conferred by larger magnet elements 16 to achieve a desired compression of the wall of the hollow organ and subsequent healing of the edges surrounding the magnet elements 16. In some implementations, different areas of the inwardly protruding portion 50 can be targeted with different sized magnet elements 16, for example, if the desired compression of one section of the inwardly protruding portion 50 differs from another section.

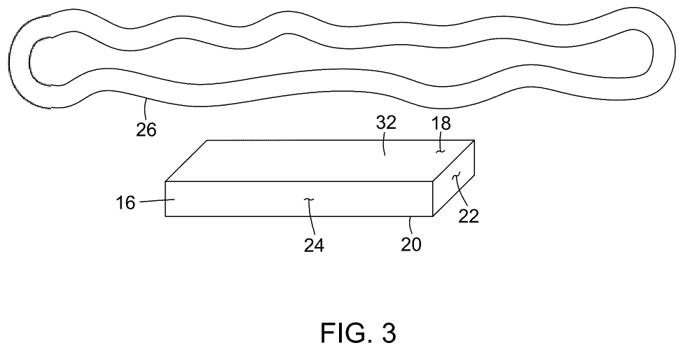

In addition, although the magnet elements 16 of a portion of a magnet assembly 12 exemplified in FIGS. 3 and 11 to 16 are represented as being substantially rectangular, it is to be understood that the magnet elements 16 can include tapered edges, chamfered edges, beveled edges, rounded edges, and the like. The magnet elements 16 can also have an oblong shape, a stadium shape, a circular shape, a triangular shape, a rectangular shape, an octagonal shape or any other shape suitable for applications described herein. In some implementations, the magnet elements 16 can have a unique shape that is configured to mate with an opposing magnet element and/or the surface of the base of the inwardly protruding portion.

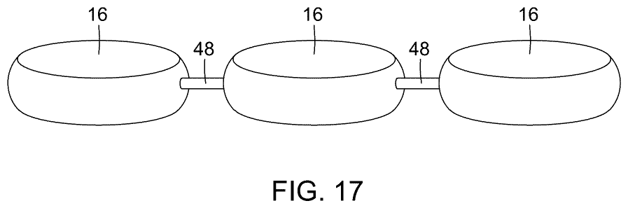

For instance, FIG. 17 illustrates an example of a series of magnet elements 16 of a portion of a magnet assembly 12, each magnet element 16 having rounded edges and a substantially oval shape. In some implementations, magnet elements 16 having rounded edges or other non-sharp features can contribute to facilitate movement between adjacent magnet elements 16 and thus provide an enhanced flexibility to the magnet assembly 12.

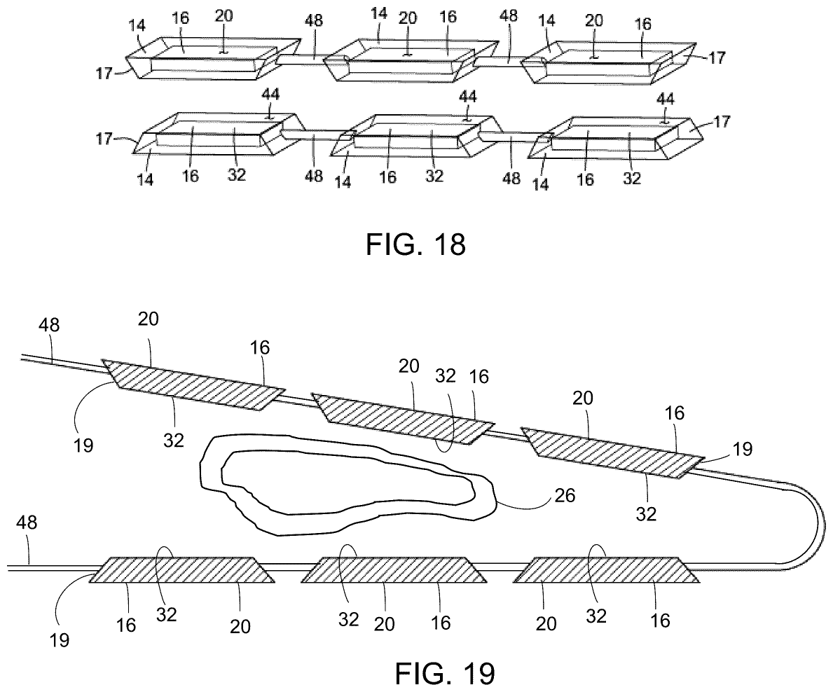

FIG. 18 illustrates an example of a series of magnet elements 16 of a portion of a magnet assembly 12, each magnet element 16 being received in a housing having beveled edges 17, the magnet elements 16 being shown facing each other as would be the case when placed on either side of the walls of the inwardly protruding portion 50.

FIG. 19 illustrates an example of magnet elements 16 of a portion of a magnet assembly 12, each magnet element 16 having beveled edges 19. In some implementations and as illustrated in FIGS. 18 and 19, when magnet elements 16 or a housing 14 include beveled edges, the magnet-engaging side 32 of the magnet element 16 or the organ-contacting side 44 of the housing 14 can have a smaller surface area compared to the side located further away from the organ tissue 26 at the base of the inwardly protruding portion, i.e., the bottom side 20. This configuration can allow for a smaller compression surface between magnet-engaging sides 32 of opposite magnet elements 16 or between organ-contacting sides 44 of opposite housings 14, such that if the ischemic necrosis was to occur more rapidly than initially foreseen, the side of the magnet elements 16 or housings 14 having a larger surface area can provide an anchor to prevent undesired movements of the magnet elements 16 or housing 14. In some implementations, the side of the magnet elements 16 or housing 14 having a larger surface area can also contribute to prevent the magnet elements 16 or housing 14 from passing through an area of the walls of the base of the inwardly protruding portion where ischemic necrosis has occurred.

In some implementations, the first zone 38 and the second zone 40 of the magnet assembly 12 can each include a single magnet element 16 that can optionally be flexibly, elastically, pivotally and/or releasably coupled to each other at one end thereof in a first transition zone. In some instances, the single magnet elements 16 of the first and second zones 38, 40 can be magnet elements that, when magnetically coupled around the base of the inwardly protruding portion, can act as a clamp to allow the opposite walls of the inwardly protruding portion to be maintained in close proximity and magnetically compressed such that ischemic pressure necrosis can occur over time, which in turn can enable the opposite walls to fuse together. In some implementations, the first and second zones 38, 40 can be flexibly, elastically, pivotally and/or releasably coupled to each other at the other ends thereof in a second transition zone.

In some implementations, the magnet-engaging side 32 of the magnet elements 16 included in the first zone 38 of the magnet assembly 12 can have a complimentary shape with regard to the magnet-engaging side 32 of the magnet elements 16 included in the second zone 40 of the magnet assembly 12. For instance, the magnet-engaging side 32 of the magnet elements 16 included in the first zone 38 can include a recess, and the magnet-engaging side 32 of the magnet elements 16 included in the second zone 40 can include a projection configured to fit within the recess. In some implementations, the engagement of the recess with a corresponding projection, or of other features having complimentary shape, can contribute to stabilize the magnet elements 16 of the first zone 38 with those of the second zone 40. In some implementations, the edges of the magnet elements 16 included in the first zone 38 of the magnet assembly 12 can include a rabbeted edge, and the edges of the magnet elements 16 included in the second zone 40 of the magnet assembly 12 can include a rabbeted edge that is complimentary to the one of the magnet elements 16 of the first zone 38.

In some implementations and as mentioned above, the attractive force of the magnet elements 16 can be chosen according to the thickness and/or to the composition of the target hollow organ along the clamp line 42. For instance, the attractive force of the magnet elements 16 can be proportional to the thickness of the target hollow organ wall. This principle can apply from one target hollow organ to another, or within the same target hollow organ. For example, the wall of the stomach being thicker than the wall of the duodenum or of another portion of the small intestine, the attractive force of the magnet elements 16 for a magnet assembly 12 designed for the stomach may be higher than for a magnet assembly 12 designed for the duodenum.

When the wall thickness varies within the same target hollow organ, the attractive force of the magnet elements 16 can vary within the first zone 38 and accordingly within the second zone 40 such that the pressure exerted on the organ tissue 26 of the target hollow organ at the base of the inwardly protruding portion (i.e., along clamp line 42) along the length of the magnet assembly 12 can also vary when in the clamping configuration. For instance, when the target hollow organ is the stomach, the wall thickness of the stomach is larger near the fundus compared to the wall thickness in the body or the antrum of the stomach. Accordingly, if the inwardly protruding portion comprises the organ tissue 26 from both areas of the stomach, the magnet elements 16 of the magnet assembly 12 can be chosen to have a higher attractive force in a portion of the magnet assembly 12 of the magnet assembly 12 that will be in contact with the wall of the fundus, compared to the attractive force of the magnet elements 16 of the magnet assembly 12 that will be in contact with the wall of the antrum.

In implementations where the target hollow organ is mainly composed of muscular fibers, such as the distal colon or the rectum, the attractive force of the magnet elements 16 may be chosen to be higher than the attractive force of the magnet elements 16 for a magnet assembly 12 designed for portions of the small intestine such as the duodenum, the jejunum or the ileum.

The attractive force of the magnet elements 16 can also be chosen so as to facilitate placement and implantation of the clamping device 10 within the lumen of the target hollow organ. For instance, in some implementations, the attractive force between the magnet elements 16 can be sufficiently weak to enable magnetic uncoupling of the magnet elements 16 during the placement of the clamping device 10 along the clamp line 42, such that trial and error may be possible to arrive at the desired location of the magnet assembly 12.

Furthermore, the attractive force of the magnet elements 16 can also be chosen in function of the planned healing period. For example, the attractive force between the magnet elements 16 can be sufficiently weak to favor a healing period in a range of days or weeks. A longer healing period can facilitate the obtention of well-fused walls and a progressive application of pressure along the clamping line 42, whereas magnet elements 16 that would have a too strong attractive force could cause rapid ischemic pressure necrosis between the magnet-engaging sides 32 of the magnet elements 16, which could impair the healing mechanism on the edges of the magnet elements 16 and could result in an opening in the tissue of the target hollow organ. In some implementations, the attractive force of the magnet elements 16 can be chosen such that the healing process occurs over a period of between 2 to 7 weeks following implantation of the clamping device 10. In some implementations, the attractive force of the magnet elements 16 is chosen such that the healing process occurs over a period of between 3 to 6 weeks following implantation of the clamping device 10. Other durations of healing periods are also possible depending on the desired result and the target hollow organ.

It is to be noted that in some implementations, the ischemic pressure necrosis to which is subjected the wall of the target hollow organ along the clamp line 42 may be sufficient to lead to a resectioning of the inwardly protruding portion 50 from the target hollow organ along the clamp line 42 with the fused tissue on the edges of the magnet elements 16. In such cases, an additional procedure may be implemented to separate and remove the inwardly protruding portion of the target hollow organ, for instance at the same time as proceeding with the removal of the clamping device 10. In other implementations, the ischemic pressure necrosis to which the wall of the target hollow organ is subjected to along the clamp line 42 may be sufficient to lead to a separation of the inwardly protruding portion of the target hollow organ, such that the severed inwardly protruding portion can be naturally excreted, for instance. The attractive force of the magnet elements 16 can thus also be chosen according to whether or not the natural severance of the inwardly protruding portion from the target hollow organ is desired.

Monitoring the healing process through time also plays a role in the result obtained on the target hollow organ following the implantation of the clamping device 10, and such monitoring can be helpful in determining at which moment to proceed with the removal of the clamping device 10 to obtain the desired result with regard to the separation of the inwardly protruding portion of the target hollow organ.

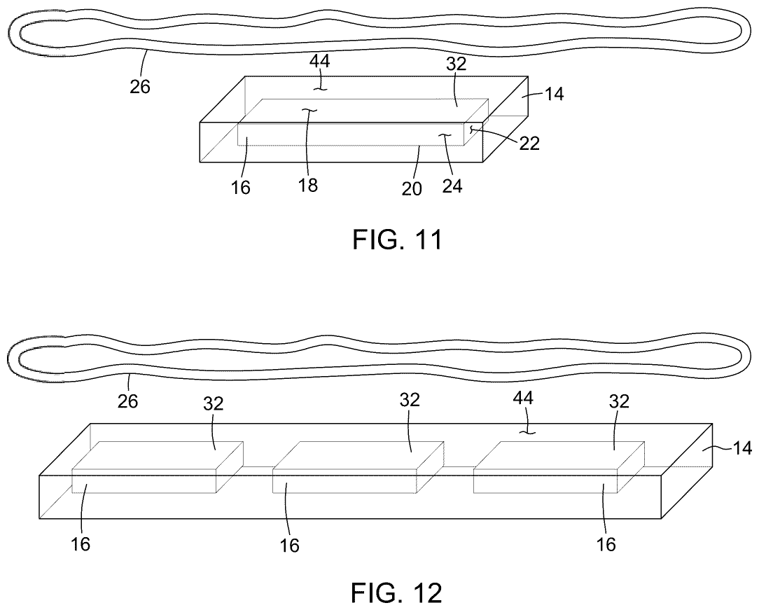

In some implementations, the size of the magnet elements 16 can vary along the length of the magnet assembly 12. This variation in the size of the magnet can contribute to increase the flexibility of the magnet assembly 12 in selected portions thereof, in particular when the magnet assembly 12 includes multiple magnet elements 16 individually received in a housing 14, such as shown in FIGS. 11, 15, and 18, or without a housing, such as shown in FIGS. 14, 17, and 19. For instance, the magnet assembly 12 can include smaller magnet elements 16 in proximity of the transition from the first zone 38 to the second zone 40. Smaller magnet elements 16 provided in proximity of this transition zone can contribute to facilitate the adoption of the looped pre-clamping configuration and the clamping configuration, by facilitating the folding over of the magnet assembly 12 such as shown in FIG. 7. Smaller magnet elements 16 in proximity of the transition from the first zone 38 to the second zone 40 can thus facilitate the placement of the clamping device 10. In some implementations, larger magnet implants, especially in terms of length, away from this transition zone can facilitate the obtention of a continuous clamp line 42 at the base of the inwardly protruding portion 50.

### Housing

With reference to FIGS. 11 to 16 and 18, in some implementations, the magnet elements 16 can be received in a housing 14. In FIG. 11, a single magnet element 16 is received in a corresponding housing 14, while in FIG. 12, multiple magnet elements 16 are received in a single housing 14. In other implementations, a certain number of magnet elements 16 can be received in a housing 16. For instance, 2 to 10 magnet elements 16 can be received in a single housing 14. In such implementations, the number of magnet elements 16 in the housing 14 can be influenced by their size, i.e., the smaller the magnet elements 16, the more successive magnet elements 16 can be received in a housing 14 having a given length.

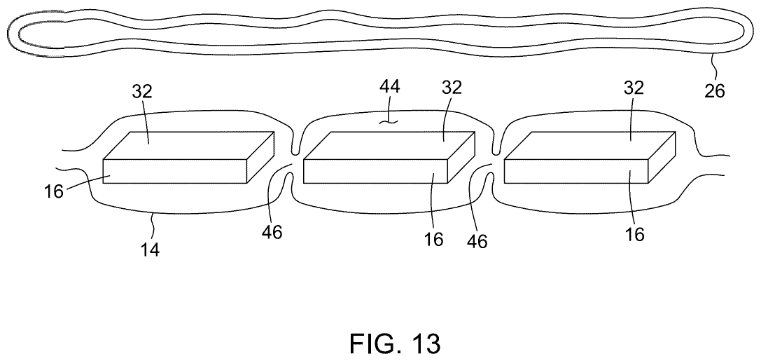

As mentioned above, the housing 14 includes an organ-contacting side 44 that is in contact with the tissue of the target hollow organ when the clamping device 10 is in the clamping configuration. In some implementations, the organ-contacting side 44 of the housing 14 includes an elongated flat contact surface to facilitate an even contact with the tissue compressed therebetween. The housing 14 can be configured to provide an atraumatic surface which can contribute to avoid damage to surrounding tissues. The housing 14 can include rounded edges, and/or tapered transitions between its walls. In some implementations, the housing 14 can have an oblong shape or a stadium shape. FIG. 13 illustrates an example of magnet elements 16 received in a respective housing 14 having rounded edges, the successive housings 14 being flexibly joined together by a thinner extension 46 of the housing 14, also referred to as a connecting portion of the housing 14. Additional details are provided below regarding this aspect.

In some implementations, the organ-contacting side 44 of the housing 14 of the magnet elements 16 included in the first zone 38 can include a recess, and the organ-contacting side 44 of the housing 14 of the magnet elements 16 included in the second zone 40 can include a projection configured to fit within the recess. The engagement of the recess with a corresponding projection, or of other features having complimentary shape, via the housings 14 can contribute to stabilize the magnet elements 16 of the first zone 38 with those of the second zone 40. In some implementations, the edges of the organ-contacting side 44 of the housing 14 of the magnet elements 16 included in the first zone 38 of the magnet assembly 12 can include a rabbeted edge, and the edges of the organ-contacting side 44 of the housing 14 of the magnet elements 16 included in the second zone 40 of the magnet assembly 12 can include a rabbeted edge that is complimentary to the one of the housings 14 of the first zone 38.

In some implementations, the housing 14 can contribute to spreading the force applied by the magnet element(s) 16 over a larger area, which can be advantageous depending on the application and/or on the target hollow organ. The housing 14 can be made of a biocompatible material. In some implementations, the housing 14 can be made of a metal such as stainless steel, titanium, or other medical implant grade metals. Alternatively, the housing 14 may be made of silicone, or other medical implant grade polymers. In certain scenarios, the housing 14 is made of Silastic™, which is a flexible silicone elastomer. In some implementations, the housing 14 can provide a protective coating around the magnet element(s) 16, which in turn can help prevent corrosion and maintain the integrity of the magnet element 16. The texture of the housing 14, in particular on the organ-contacting side 44, can be chosen to provide an increased stability to the magnet assembly 12 once in the clamping configuration. For instance, the surface roughness of the implant on the organ-contacting side 44 of the housing 14 can be higher than on the opposite side thereof. The surface roughness of the organ-contacting side 44 of the housing 14 may be beneficial once the magnet elements 16 are magnetically coupled to prevent magnetic decoupling due to shear forces.

### Connection Between Adjacent Magnet Elements

As mentioned above, the magnet assembly 12 includes magnet elements 16 that are connected to each other in series. In some implementations, the magnet elements 16 are flexibly connected to each other in series. Flexibly connecting the magnet elements 16 together can be achieved in various ways. Examples are provided below. In other implementations, some or all of the magnet elements 16 in the same zone (i.e., having the same polarity) may have a rigid or semi-rigid connection between adjacent magnet elements to facilitate a clamping, flattening, or compression of the base of the inwardly protruding portion.

With reference to FIG. 13, multiple magnet elements 16 can be received in a respective single housing 14, and the housing 14 can include portions in between adjacent magnet elements 16 that are adapted to provide an increased flexibility to the magnet assembly 12. As shown in FIG. 13, these portions 46 can be narrower, or thinner, to increase their freedom of movement, while remaining sufficiently robust to avoid breaking. In some implementations, the portion 46 of the housing 14 between adjacent magnet elements 16 can be made of a material that is different from the material of the rest of the housing 14, to benefit from specific characteristics of this different material, in particular in terms of its flexibly. In some implementations, the portion 46 of the housing 14 between adjacent magnet elements 16 can offer a certain range of motion in a certain plan, while offering a restricted range of motion in another plan. For example, it may be desired to have the magnet elements 16 move relative to each other to enable the magnet assembly 12 to fold around the base of the inwardly protruding portion, while it may be less desirable to have the magnet elements 16 move laterally relative to each other, as lateral movement of the magnet elements 16 could contribute to veering the magnet elements 16 off of the clamp line 42.

With reference to FIG. 14, when no housing is provided around the magnet elements 16, the magnet elements 16 can include a hook (not shown) on each lateral side thereof, and be connected to each other via a flexible connector 48, such as a string or elastic. The type of flexible connector 48 can be chosen to provide sufficient mobility and flexibility to the magnet assembly 12 according to a given range of movement, so that the magnet assembly 12 can easily be guided to its destination in the lumen of the hollow organ and placed at the base of the inwardly protruding portion. For example, when it is desired to severe the appendix, for instance in cases of appendicitis, the magnet assembly 12 can be positioned in the lumen of the large intestine as the target hollow organ, which has a relatively smaller size compared to the stomach. Accordingly, it may be beneficial to have a magnet assembly 12 having an increased flexibility to facilitate guiding the clamping device 10 within the lumen of the large intestine and maintaining it in place around the base of the appendix once reverted as an inwardly protruding portion within the lumen of the large intestine.

With reference to FIG. 15, a flexible connector 48 can be provided between adjacent magnet elements 16 that are individually housed in a respective housing 14. In such implementations, an engaging portion, such as a hook, can be provided on the lateral sides of the housing 14. In some implementations, the engaging portion can be integral with the housing 14. The housing 14 can thus be molded as a single piece unit that includes a hook on each lateral side thereof, with the magnet element 16 received in the housing 14, and adjacent ones of the multiple magnet elements 16 can be flexibly connected to each other via a flexible connector 48 engaged with respective hooks. Alternatively, a hook can be provided on a lateral side of the housing 14, and a flexible connector 48 can extend from another side to flexibly connect with a hook of an adjacent housing 14.

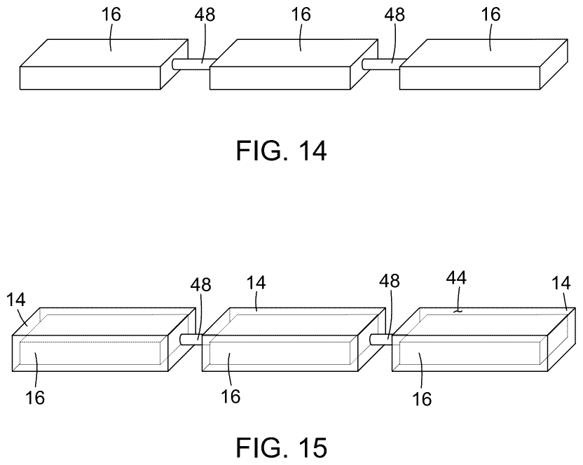

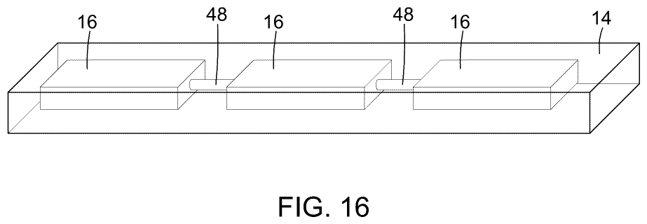

In other implementations and with reference to FIG. 16, the magnet elements 16 can be flexibly connected to each other by being integrated within the housing 14, and a flexible connector 48 can also be provided between adjacent magnet elements 16 within the housing 14. In the implementation shown in FIG. 16, in contrast to the implementation shown in FIG. 15, the flexible connectors 48 are thus shown integrated within the housing 14.

It is to be understood and as shown in FIG. 12, that the flexibility of the magnet assembly 12 can be provided by the presence of the housing 14 itself around the magnet elements 16, for instance given the flexibility of the material from which is made the housing 14. In other implementations, the rigidity of the magnet assembly 12 can be provided by the housing 14, for instance given the rigidity of the material the housing is 14 made from.

The distance between adjacent magnet elements 16 can be chosen so as to have an impact on the resulting flexibility, or alternatively rigidity, of the magnet assembly 12. In some implementations, when determining the distance between adjacent magnet elements 16, care should be taken to maintain an equilibrium between the flexibility of the magnet assembly 12 obtained in such manner and the impact on the resulting clamp line 42, as successive magnet elements 16 that may be too distant apart from each other may result in a less uniform clamp line 42. In some implementations, the distance between adjacent magnet elements 16 can vary along the length of the magnet assembly 12, similarly to what is described above regarding the size of the magnet elements 16. For instance, in some implementations, magnet elements 16 located in proximity of the transition from the first zone 38 to the second zone 40 may be provided slightly further apart to provide enhanced flexibility to the magnet assembly 12 in this transitioning area. In some implementations, the interplay between the size of the magnet elements 16 and the distance between adjacent magnet elements 16 can contribute to provide an enhanced flexibility to the magnet assembly 12 and a desired clamp line 42. In some implementations, magnet elements 16 provided as close as possible to each other can be favored as long as flexibility of the magnet assembly 12 is preserved. In that respect and as mentioned above, magnet elements 16 or housings 14 having rounded edges can be advantageous to provide magnet elements 16 or housings 14 close together but without compromising flexibility of the magnet assembly 12. It is to be noted that these are examples only to illustrate the effect that the various configurations may have on the flexibility of the magnet assembly 12, and that multiple configurations of the magnet assembly 12 can be implemented to achieve a combination of a desired flexibility of the magnet assembly 12 and a desired clamp line 42 and corresponding healing of the tissue on the edges of the magnet elements 16.

### Fasteners and Connectors to Form an Enclosed Clamping Device

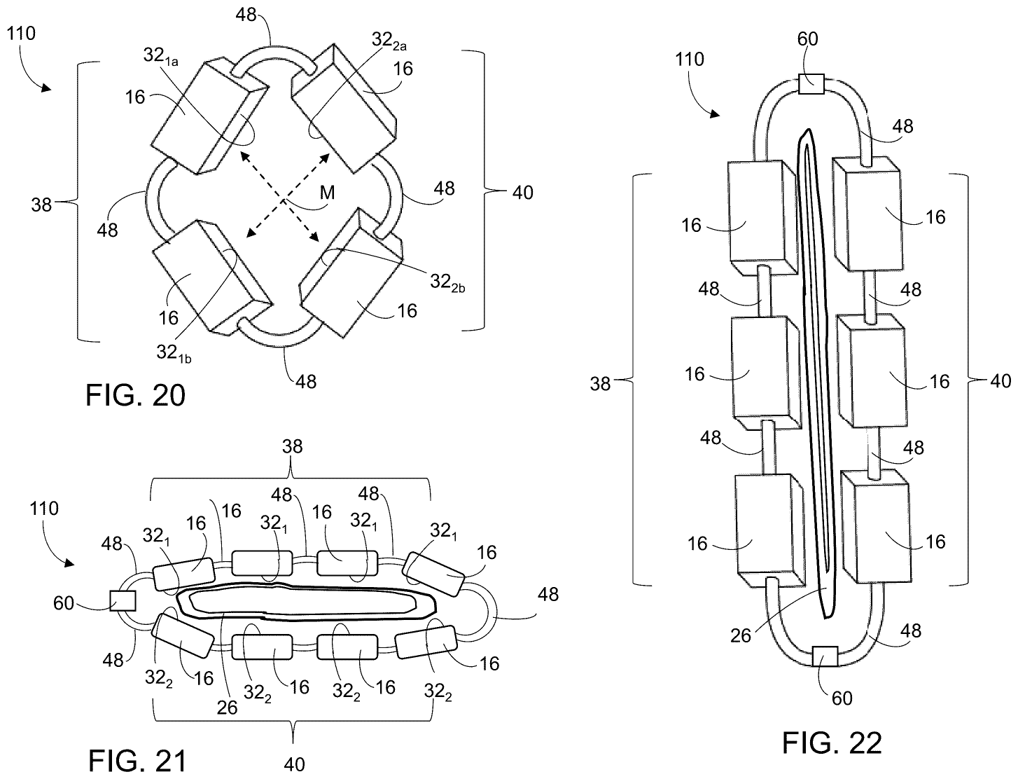

In other implementations and with reference to FIGS. 20 to 22, the clamping device can be an enclosed clamping device 110. The enclosed clamping device 110 includes a first zone 38 of magnet elements 16 and a second zone 40 of magnet elements 16. The first zone 38 and the second zone 40 can be flexibly coupled with flexible connectors 48, thus providing first and second transition zones. In such implementations, the enclosed clamping device 110 forms an enclosed shape when in the pre-clamping configuration, which can also be referred to as an enclosed pre-clamping configuration. When in use, the inwardly protruding portion of the target hollow organ can be pulled through the opening of the enclosed clamping device 110 in the enclosed pre-clamping configuration, such that the enclosed clamping device 110 can be positioned around the base of the inwardly protruding portion along a clamp line.

When in the clamping configuration, a magnet-engaging side 321 of the first zone 38 couples with a corresponding magnet-engaging side 322 of the second zone 40, such that the enclosed clamping device 110 can form an elongated shape around the organ tissue 26 of the base of the inwardly protruding portion, such as shown in FIGS. 21 and 22. Over time, the pressure exerted on the wall of the target hollow organ causes ischemic pressure necrosis at the base of the inwardly protruding portion, resulting in the fusion of the two layers forming the fold, i.e., forming the inwardly protruding portion, at the base thereof. Over time, this fusion can also result in the severance of the inwardly protruding portion from the remainder of the hollow organ, or the inwardly protruding portion can be resectioned in a secondary procedure when removing the enclosed clamping device 110. In such configurations, the base of the inwardly protruding portion can be considered as being elongated, and forming a substantially oval shape, as shown in FIG. 22.

With specific reference to FIG. 20, in some implementations, the clamping device 110 can be configured such that a magnet element 16 in the first zone 38 that is closest to the first or second transition zone magnetically couples with a corresponding magnet element 16 in the second zone 40. In other words, a magnetic-coupling side 321a of a magnet element 16 in the first zone 38 near the first transition zone magnetically couples with a magnetic-coupling side 322a of a magnet element 16 in the second zone 40 near the first transition zone. In other implementations, the clamping device 110 can be configured such that opposed magnet elements 16 are magnetically attracted M to each other towards a center of the opening in the enclosed pre-clamping configuration. In other words, the magnetic-coupling side 321a of a magnet element 16 in the first zone 38 near the first transition zone can be configured to magnetically couple a magnetic-coupling side 322b of a magnet element 16 in the second zone 40 near the second transition zone and vice versa (magnetic-coupling side 321b being magnetically attracted to magnetic-coupling side 322a). In such an implementation, the magnetic attraction M is directed towards a center of the base of the inwardly protruding portion, such that the base of the inwardly protruding portion forms a shape that is substantially circular, oval, or elliptical.

In some implementations, the first zone 38 and the second zone 40 can be releasably coupled at one end thereof, such as shown in FIG. 21, or releasably coupled at both ends thereof, such as shown in FIG. 22. In some implementations, the first zone 38 and the second zone 40 are releasably coupled with a fastener 60. The fastener 60 can be any known means of fastening two portions together, such as a hook and loop or clasps, including magnetic clasps, bolo clasp, barrel clasp, box clasp, bead and ball clasp, fishhook clasp, hook clasp, etc. Therefore, the flexibility of the magnet assembly in the clamping device 110 can, in part, be provided by the flexible connectors 48 and the releasable fastener 60.

With specific reference to FIG. 21, the first zone 38 and the second zone 40 can be releasably coupled at a first transition zone with the flexible connector 48 and coupled at a second transition zone with a fastener 60, such that the device forms an enclosed pre-clamping configuration. Alternatively, the fastener 60 at the second transition zone can be released, such that the first zone 38 and the second zone 40 are not coupled at the second transition zone. When the fastener 60 at the second transition zone is released, the clamping device 110 can be in a looped pre-clamping configuration or an extended pre-clamping configuration, depending on the method of inserting the clamping device 110 into the lumen of the target hollow organ, the nature of the procedure, the type of target hollow organ, etc. Once the clamping device 110 is positioned around the organ tissue 26 of the base of the inwardly protruding portion, the fastener 60 can be used to couple together the first zone 38 and the second zone 40 at the second transition zone to place the clamping device 110 in the enclosed pre-clamping configuration, and the magnetic-coupling sides 321, 322 of the first zone 38 and the second zone 40, respectively, can be magnetically coupled to place the clamping device 110 in the clamping configuration.

With specific reference to FIG. 22, the first zone 38 and the second zone 40 can be coupled at a first transition zone and a second transition zone with a corresponding fastener 60, such that the device is in the enclosed pre-clamping configuration. The fastener 60 at either the first or the second transition zone can be configured to be released, such that the first zone 38 and the second zone 40 are not coupled at one end and the clamping device can be in a looped pre-clamping configuration or an extended pre-clamping configuration. In some implementations, the first zone 38 and the second zone 40 can be releasably uncoupled at both the first transition zone and the second transition zone, such that the clamping device 110 can be in an uncoupled pre-clamping configuration. The uncoupled pre-clamping configuration can facilitate an easier maneuvering of the clamping device 110 around the base of the inwardly protruding portion, and then the fasteners 60 at the first and second transition zones can be coupled to position the clamping device 110 in the enclosed pre-clamping configuration, and the magnetic-coupling sides 321, 322 of the first zone 38 and the second zone 40, respectively, can be magnetically coupled to place the clamping device 110 in the clamping configuration.

In some implementations, the flexible connectors 48 between adjacent magnet elements 16 of the same zone (i.e., the first zone 38 or the second zone 40) can be more rigid than the flexible connectors 48 coupling the first zone 38 and second zone 40 to each other or to the fasteners 60. In such implementations, the flexible connectors 48 at the first transition zone can act as a hinge, whereby the rigid-like configurations of the first zone 38 and the second zone 40 can act to contribute to flatten or elongate the base of the inwardly protruding 50 until the fastener 60 at the second transition zone can be fastened. In such implementations, the clamping device 110 can function in part as a clip or clamp to clamp the base upon the coupling of the fastener 60 and stabilize the magnet assembly 12 at the base of the inwardly protruding portion.

### Positioning and Implantation of the Clamping Device

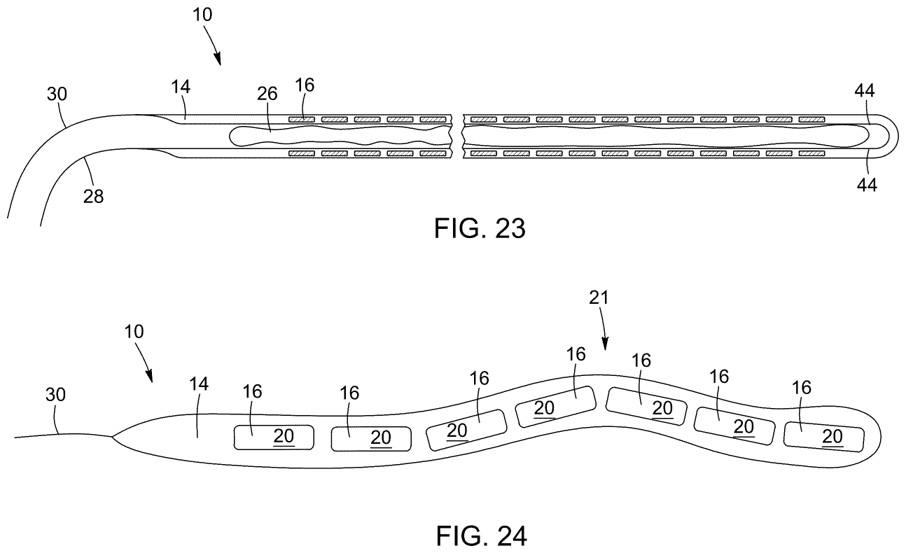

With reference now to FIGS. 23 to 29C, implementations related to the implantation of the clamping device at various locations for a given target hollow organ, and for different target hollow organs, will be now described.

FIG. 23 illustrates an example of a clamping device 10 in a clamping configuration, with the organ tissue 26 at the base of an inwardly protruding portion being compressed between the organ-contacting sides 44 of the housing 14, and the magnet-engaging sides 32 of the magnet elements 16 from the first zone 38 being magnetically coupled to the magnet-engaging sides 32 of the magnet elements 16 from the second zone 40. The clamping device 10 can be implanted along a desired clamp line 42 (i.e., at the base of the inwardly protruding portion).

Referring back to FIGS. 6 to 10, an example of a clamp line 42 (shown more specifically in FIG. 9) that extends around the base of an inwardly protruding portion of the stomach is shown. The location of the clamp line 42 can vary depending on the volume of the stomach that is desired to be obtained or the location of the tumor to be removed. In such implementations, the clamping device 10 can be placed at the base of the inwardly protruding portion by inserting the clamping device 10 into the lumen of the stomach in an extended pre-clamping configuration, such as shown in FIG. 6, or in a loop pre-clamping configuration, such as shown in FIG. 7. In some implementations, the clamping device 10 is placed inside the lumen in the extended pre-clamping configuration and then placed in the loop pre-clamping configuration or the enclosed pre-clamping configuration. In other implementations, the clamping device 10 can be placed inside the lumen in the uncoupled pre-clamping configuration or the enclosed pre-clamping configuration. The insertion and guiding of the clamping device 10 can be done endoscopically for a minimally invasive procedure, for example with an upper endoscopy or a colonoscopy. The extended pre-clamping configuration or the uncoupled pre-clamping configuration can facilitate easier insertion of the clamping device 10 into the lumen.

In implementations where the clamping device is used to reduce the volume of a target hollow organ, a selected portion of the organ that is intended to be folded in to form an inwardly protruding portion is first identified, and then the selected portion of the organ is pulled into the lumen of the target hollow organ with a surgical tool 52. In some implementations, the inwardly protruding portion 50 can be drawn or pulled into the lumen before or after the clamping device 10 is inserted into the lumen of the organ. For example, the inwardly protruding portion 50 can be drawn into the lumen and clamped with any known type of surgical clamp and then the clamping device 10 can be inserted into the lumen. In other implementations, the clamping device 10 can be inserted into the lumen, and then the inwardly protruding portion 50 can be drawn into the lumen and immediately clamped or compressed at the base with the clamping device 10. The inwardly protruding portion 50 can be drawn or pulled into the lumen with a surgical tool 52 for grasping and moving the wall of the organ, such as endoscopic grasper jaws, a flexible spring plunger, a spring claw, a mechanical pickup, or forceps, including, without limitation, adson bipolar forceps, adson brown forceps, babock forceps, carmalt forceps, etc.

When inserting the clamping device 10 into the lumen in the extended pre-clamping configuration, the clamping device 10 can be navigated around the base of the inwardly protruding portion 50, and aligned with the desired clamp line 42. Once a proper alignment of the clamping device 10 is obtained, the magnet elements 16 of the first zone 38 of the magnet assembly 12 can be brought in sufficiently close proximity of the magnet elements 16 of the second zone 40 of the magnet assembly 12 such that the magnet-engaging sides 32 of the magnet elements 16 of the first zone 38 can be magnetically coupled to the magnet-engaging sides 32 of the magnet elements 16 of the second zone 40. As mentioned above, the attractive force of the magnet elements 16 can be chosen to enable trial and error until proper placement of the magnet assembly 12 is achieved.

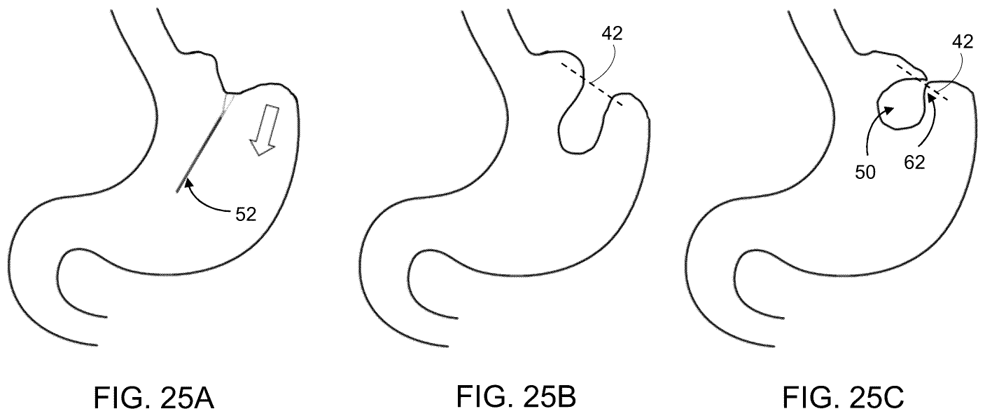

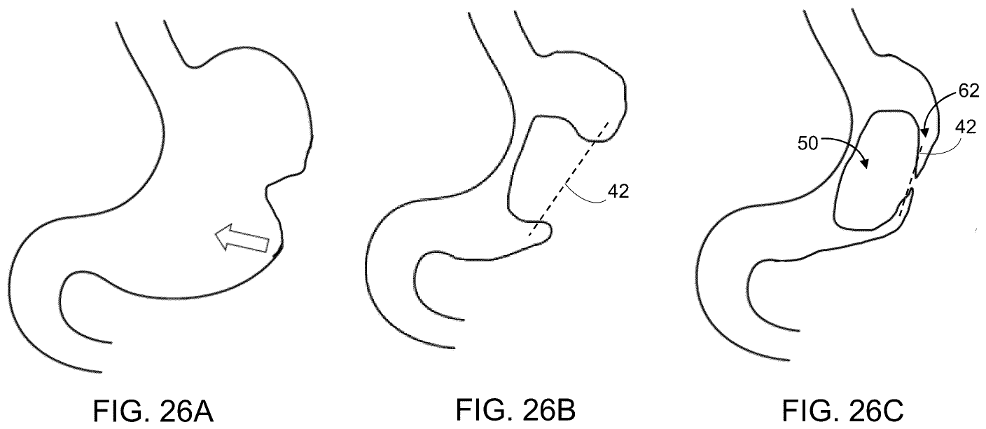

Referring to FIGS. 25A to 25C, in some implementations, the clamp line 42 can be placed at an upper portion of the stomach, such that at least a portion of the fundus of the stomach is folded in to form the inwardly protruding portion 50. Referring to FIGS. 26A to 26C, in some implementations, the clamp line 42, can be positioned along the vertical side wall of the body of the stomach, such that the clamp line 42 is similar to that of a vertical sleeve gastrectomy, for instance. In both implementations, the formation of the inwardly protruding portion 50 reduces the volume of the lumen of the stomach. Once the walls of the stomach forming the inwardly protruding portion have fused together at the base of the inwardly protruding portion, i.e., in between the magnet elements, the inwardly protruding portion 50 can remain in the lumen of the stomach for a given period of time, or can be removed in a secondary procedure, for example when removing the clamping device 10 from the lumen of the stomach endoscopically.

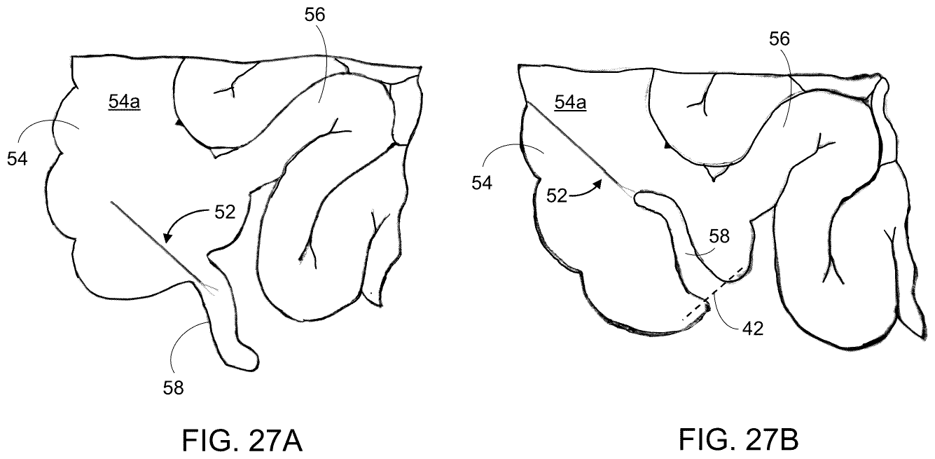

Referring now to FIGS. 27A and 27B, a partial view of an abdominal cavity including a large intestine 54, a small intestine 56, and an appendix 58 is shown. In some implementations, the clamping device 10 can be used in an appendectomy procedure (i.e., the resectioning of the appendix 58). In such implementations, a surgical tool 52 is inserted into the lumen 54a of the large intestine 54, for example through an endoscopic procedure. The surgical tool 52 is then used to pull or draw in the appendix 58 into the lumen 54a of the large intestine 54. The clamping device 10, which can be inserted into the lumen 54a of the large intestine 54 either before or after the appendix 58 is drawn or pulled into the lumen 54a, can then be positioned around the base of the appendix 58 at the clamp line 42. In such implementations, the appendix 58 can be considered as corresponding to an inwardly protruding portion of the large intestine, and the clamping device 10 can be used to resection the appendix 58 via ischemic pressure necrosis. Once the organ tissue 26 around the base of the appendix 58 (i.e., the tissue at the clamp line 42) has fused together, the appendix 58 and the clamping device 10 can be removed in a secondary procedure.

In other implementations, the clamping device 10 can be used to remove or resection a portion of the inner wall of a target hollow organ. In such implementations, the portion of the organ can be removed or resectioned to remove for instance a tumor, lesion, necrotic tissue, etc. from the wall of the target organ. Alternatively, when the target hollow organ has two or more types of wall tissue, the inwardly protruding portion 50 can comprise an inner tissue of the hollow organ. In some implementations, the inwardly protruding portion 50 can entirely or partially comprise a tumour, and the clamping device 10 can be inserted into the lumen of the target hollow organ and positioned around a base of the tumour, which would corresponding to an inwardly protruding portion of the organ.

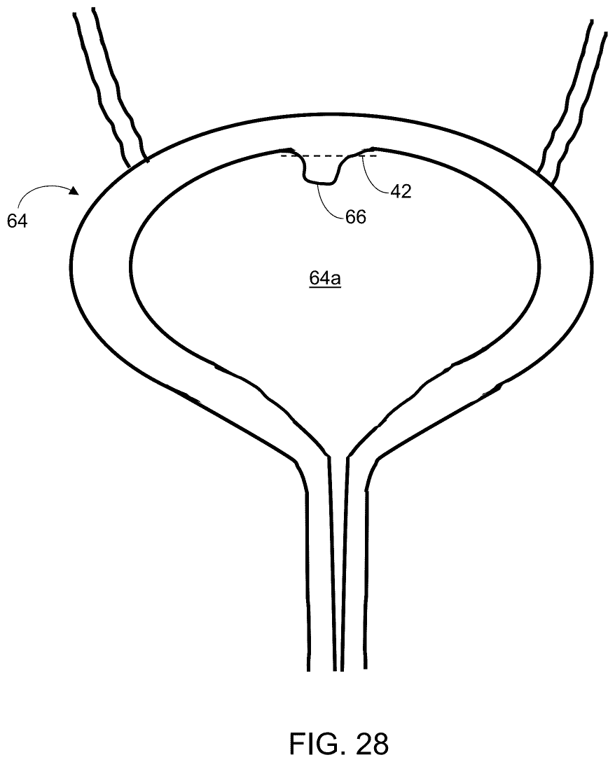

Referring now to FIG. 28, a bladder 64 of a female patient with a tumour 66 in the interior surface of the detrusor muscle is shown. In this implementation, the clamping device 10 can be inserted into the lumen 64a of the bladder 64 and placed around a base of the tumour 66 at the clamp line 42, such that the inwardly protruding portion entirely comprises the tumour 66. In some implementations, depending on the placement and depth of the tumour in the tissue lining the lumen of the bladder 64, a surgical tool 52 can be used to draw or pull the tumour 66 further into the lumen, such that the inwardly protruding portion partially comprises the tumour 66, as well as healthy or non-tumour tissue of the detrusor muscle. Once the ischemic pressure necrosis causes the detrusor muscle tissue between the magnet elements of the magnet assembly to fuse together, the tumour 66 can be resectioned and removed from the lumen 64a of the bladder 64 in a secondary procedure, such as when removing the clamping device. In such implementations, the clamping device 10 can act to increase the volume of the lumen of the target hollow organ by removing an extraneous portion, such as a tumour, that extends into the lumen of the organ.

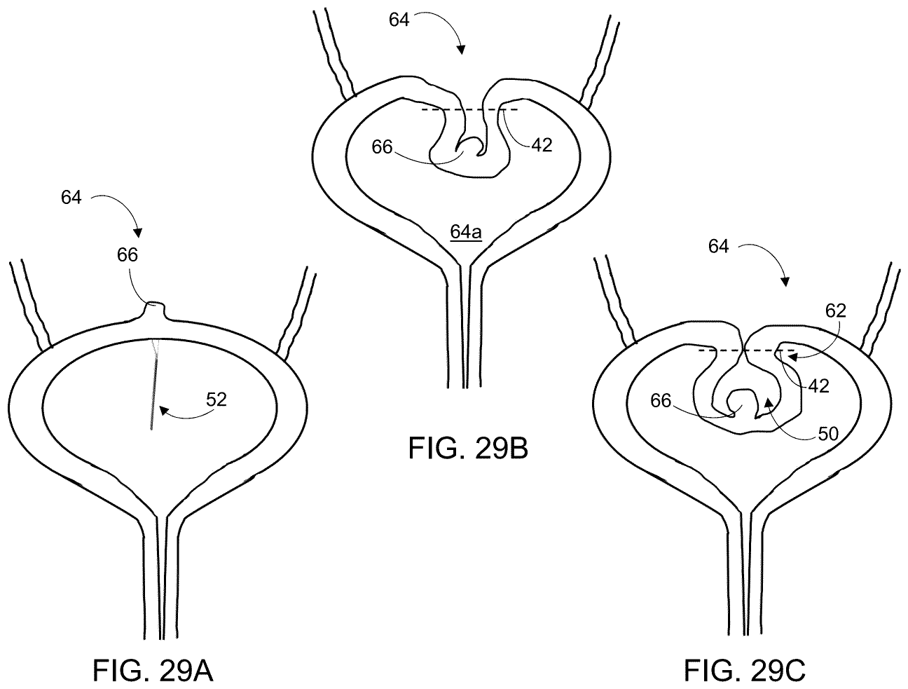

Referring now to FIGS. 29A to 29C, a bladder 64 of a female patient with a tumour 66 on an exterior wall surface of the bladder 64 is shown. In this implementation, a surgical tool 52 can be used to grasp the inner wall of the lumen 64a of the bladder 64 on an opposing side of the tumour 66. The inner wall of the lumen 64a can then be pulled inwardly into the lumen 64a of the bladder 64, such that the tumour 66 becomes part of an inwardly protruding portion 50. The clamping device 10 can be inserted into the lumen 64a of the bladder 64 and placed at the clamp line 42 to surround the base of the tumour 66. Once the ischemic pressure necrosis causes the tissue between the magnet elements of the magnet assembly to fuse together, the inwardly protruding portion 50 can be resectioned and removed from the lumen 64a of the bladder 64 in a secondary procedure, such as when removing the clamping device.

### Method for Implanting the Clamping Device

A method for resectioning a portion of a hollow organ using a clamping device as described herein will now be described in further detail.

In some implementations, the clamping device includes a magnet assembly, which in turn includes a plurality of magnet elements flexibly connected in series. In some implementations, the magnet assembly can include a fastener that enables releasable coupling of a first portion and a second portion of the magnet assembly, such as a clip, hook and loop, or clasp, such that the magnet assembly can form an enclosed shape.

The method includes inserting the clamping device into a lumen of the target hollow organ of the patient. As the clamping device is an implant that is configured to remain in the lumen of the target hollow organ of the patient for a certain period of time, the clamping device may be sterilized prior to insertion into the lumen of the hollow organ of the patient. For insertion into the lumen of the target hollow organ of the patient, the magnet assembly can be in an extended pre-clamping configuration, in a looped pre-clamping configuration, in an enclosed pre-clamping configuration, in an uncoupled pre-clamping configuration, or in a clamping configuration (i.e., the first and second zones of the clamping device being magnetically coupled, which involves magnetically uncoupling the first and second zones to place the clamping device around the base of the inwardly protruding portion). The magnet assembly can then guided, in some instances endoscopically, to position the magnet assembly around the base of an inwardly protruding portion of the hollow organ. Various techniques can be used to insert the magnet assembly into the lumen of the target hollow organ of the patient and place the magnet assembly around the base of an inwardly protruding portion of the target hollow organ. These techniques can include image-guided procedures and flexible endoscopy, for instance. When the first zone of the magnet assembly faces the second zone of the magnet assembly, the magnet-engaging sides of the magnet elements from the first zone of the magnet assembly face the magnet-engaging sides of the magnet elements from the second zone of the magnet assembly, while the magnet elements remain magnetically uncoupled in a looped pre-clamping configuration, and enclosed pre-clamping configuration, or a uncoupled pre-clamping configuration to facilitate placement of the magnet assembly along a desired clamp line (i.e. around the base of the inwardly protruding portion). When the clamping device forms a substantially circular or otherwise enclosed shape, whether as an enclosed circular shape or as a looped circular shape, the magnet elements can remain magnetically uncoupled during placement around the base of the inwardly protruding portion.

In some implementations, either before or after the clamping device is placed intra-luminally in the target hollow organ, the method can include using a surgical tool, such as forceps, to draw in or pull in a portion of the organ, or an adjacent organ, into the lumen of the target hollow organ. For example, during an appendectomy, the target hollow organ is the large intestine and the adjacent organ to be resection is the appendix. During the appendectomy, surgical tools can be used to draw the appendix into the lumen of the large intestine, and the clamping device can be placed the base of the appendix while the appendix inside the lumen of the large intestine.

In some implementations, such as during a bariatric procedure, the method can include using a surgical tool can be used to draw in or pull in a portion of the stomach tissue to form an inwardly protruding portion, and the clamping device can placed against the interior wall of the stomach at the base of the inwardly protruding portion. By drawing in or pulling in the portion of the stomach tissue to form an inwardly protruding portion, the overall volume of the stomach is reduced.

In some implementations, such as during a resection or excision of a tumour or a lesion, or other types of undesirable tissue on an interior wall of the target hollow organ, the method can include using a surgical tool to draw in or pull in the undesirable tissue, and the clamping device can be placed around the base of the inwardly protruding portion with includes the undesirable tissue, and can also comprise the margin or healthy tissue surrounding the undesirable tissue. During a resection or excision of undesirable tissue that is located on an exterior wall (extraluminal tumour) of the target hollow organ or undesirable tissue that extends through the wall of the target hollow organ, the method can include using a surgical tool to draw in or pull in the undesirable tissue within the lumen of the target hollow organ to form an inwardly protruding portion such that the undesirable tissue is enclosed with the inwardly protruding portion. It is to be noted that when the undesirable tissue extends through the wall of the target hollow organ, a portion of the outer tissue of the inwardly protruding portion can comprise the undesirable tissue.

Once the magnet elements on both sides of the magnet assembly are aligned as desired along the base of the inwardly protruding portion, (i.e., at the clamp line), the magnet elements can be brought closer together in a clamping configuration. In the clamping configuration, the magnet elements of the first zone magnetically couple magnet elements of the second zone, i.e., magnets elements that are facing each other magnetically couple, via their respective magnet-engaging sides to compress opposite walls of the inwardly protruding portion, the two layers forming the fold, therebetween. In some implementations, when the magnet elements are received in a housing, the housing includes an organ-contacting side and the opposite walls of the inwardly protruding portion are compressed between the organ-contacting sides of the housing that are facing each other. In some implementations, the organ-contacting side of the housing can be an elongated flat contact surface.

In some implementations, once the clamping device is in the clamping configuration, a fastener at one or both ends of each of the first and second zones of magnet elements can be releasably coupled, such that the clamping device can be in an enclosed clamping configuration.

Then, following the implantation of the magnet assembly around the base of the inwardly protruding portion of the target hollow organ in the clamping configuration, the magnet assembly can be left implanted for a duration of a healing period of time that is sufficient to allow the opposite walls of the inwardly protruding portion to fuse together while the magnet elements are magnetically coupled together. In some implementations, the healing period can extend from between 2 weeks to 7 weeks. In some implementations, monitoring the healing process can be performed to determine at which moment the walls of the opposite walls of the inwardly protruding portion are well fused together. In some implementations, the duration of the healing period can depend on the target hollow organ, the configuration of the magnet assembly (i.e., size, magnetic strength, and shape of the magnet elements), the condition of the patient, and various other factors.

Once the opposite walls of the inwardly protruding portion are well fused together and that healing is completed, the clamping device can be removed from the lumen of the target hollow organ. The method for removing the magnet assembly can vary depending on the design of the clamping device. In some implementations, the clamping device can be removed from the lumen of the target hollow organ and the abdominal cavity endoscopically. Again, various techniques can be used to remove the magnet assembly from the lumen of the target hollow organ of the patient, which may be the same or different as the technique used previously to insert and place the magnet assembly in the lumen of the target hollow organ, and can include image-guided procedures and flexible endoscopy, for instance.

In some implementations, such as in the context of procedures for reducing a volume of a hollow organ, e.g., bariatric procedures, the method for resectioning a portion of a hollow organ using a clamping device as described herein can be performed sequentially. For example, an initial, or first, surgical procedure can be conducted to resection a first portion of the hollow organ and thus reduce the volume of the hollow organ by a portion of the desired overall volume reduction. Once the initial surgical procedure is completed and a sufficient healing time period has passed, a subsequent, or second, surgical procedure can be conducted to resection a second portion of the hollow organ and thus reduce the volume of the hollow organ by another portion of the desired overall volume reduction, thereby further reducing the volume of the hollow organ. In such implementations, any number of successive surgical procedures, each occurring after a sufficient corresponding healing time, can be performed until the desired overall volume reduction is reached. In some implementations, the targeted area (i.e., the area that forms the inwardly protruding portion) in the subsequent surgical procedures (the second, third, etc. surgical procedure) can be different than the targeted area in the preceding surgical procedure or procedures. By performing successive volume reduction procedures, the organ can have additional time to heal and adjust to the reduced volume size, prior to reducing the volume further.

Several alternative implementations and examples have been described and illustrated herein. The implementations of the technology described above are intended to be exemplary only. A person of ordinary skill in the art would appreciate the features of the individual implementations, and the possible combinations and variations of the components. A person of ordinary skill in the art would further appreciate that any of the implementations could be provided in any combination with the other implementations disclosed herein. It is understood that the technology may be embodied in other specific forms without departing from the central characteristics thereof. The present implementations and examples, therefore, are to be considered in all respects as illustrative and not restrictive, and the technology is not to be limited to the details given herein. Accordingly, while the specific implementations have been illustrated and described, numerous modifications come to mind.

1. A clamping device for resectioning an inwardly protruding portion present in a lumen of a hollow organ of a patient, the clamping device comprising:
a magnet assembly implantable into the lumen of the hollow organ of the patient, wherein the magnet assembly comprises:
a first zone comprising a first magnet element; and
a second zone comprising a second magnet element; and
the magnet assembly being configured to be positioned at a base of the inwardly protruding portion to magnetically couple the first zone and the second zone to compress opposite walls of the inwardly protruding portion therebetween during a healing time period.

2. The clamping device claim 1, further comprising a transition zone extending between the first zone and the second zone, the transition zone comprising a fastener configured to couple a leading end of the first zone to a trailing end of the second zone.

3. The clamping device of claim 2, wherein the fastener comprises a flexible fastener.

4. The clamping device of claim 2, wherein the fastener comprises a releasable fastener.

5. The clamping device of claim 2, wherein the transition zone is configured to provide a pivotal transition between the first zone and the second zone.

6. The clamping device of claim 2, wherein the transition zone further comprises a flexible connector.

7. The clamping device of claim 1, wherein at least one of the first and second magnet elements comprises a plurality of magnet elements, the magnet elements of the plurality of magnet elements being connected to each other in series by a magnet element connector.

8. The clamping device of claim 7, wherein the magnet element connector is flexible.

9. The clamping device of claim 7, wherein the magnet element connector is a rigid or semi-rigid connector.

10. The clamping device of claim 1, wherein at least one of the first and second magnet elements comprise bevelled edges or rounded edges.

11. The clamping device of claim 1, wherein the clamping device is configurable between an uncoupled pre-clamping configuration and a clamping configuration.

12. The clamping device of claim 1, wherein the clamping device is configurable between an enclosed pre-clamping configuration and a clamping configuration.

13. The clamping device of claim 1, wherein at least one of the first and second magnet element comprises a housing comprising an organ-contacting side and being configured to receive the at least one of the first and second magnet element therein.

14. The clamping device of claim 13, wherein the organ-contacting side of the housing comprises an elongated flat contact surface.

15. The clamping device of claim 13, wherein the housing comprises bevelled edges.

16. The clamping device of claim 1, wherein the first and second magnet elements are received in a housing, the housing comprising a first end and a second end, and wherein the first end and the second end of the housing are releasably coupled to each other with a fastener.

17. The clamping device of claim 1, wherein the first and second magnet elements are received in a corresponding housing.

18. The clamping device of claim 1, wherein an attractive force of the first and second magnet elements is determined at least in part in accordance with a thickness and/or a composition of the opposite walls of the inwardly protruding portion.

19. The clamping device of claim 1, wherein an attractive force of the first and second magnet elements is determined so as to facilitate placement and implantation of the clamping device around the portion of the inwardly protruding portion.

20. A method for resectioning an inwardly protruding portion present in a hollow organ of a patient, the method comprising the steps of:
inserting a magnet assembly of a clamping device into a lumen of the hollow organ, the magnet assembly comprising a first zone and a second zone, each of the first zone and the second zone comprising at least one magnet element having a magnetic-engaging side;
positioning the magnet assembly at a base of the inwardly protruding portion such that the magnetic-engaging side of the first zone and the magnet-engaging side of the second zone face each other;
magnetically coupling the first zone and the second zone together to compress opposite walls of the inwardly protruding portion therebetween; and
allowing the opposite walls of the inwardly protruding portion to fuse together via ischemic pressure necrosis while the first zone and the second zone remain magnetically coupled during a healing time period.
